(19) United States
(12) Patent Application Publication
(10) Pub. No.: US 2010/0279383 A1
(43) Pub. Date: Nov. 4, 2010

(54) Subtilases and subtilase variants having altered immunogenicity

(75) Inventors: Erwin Ludo Roggen, Lyngby (DK); Nina Teeres Herne, Stockholm (SE); Steffen Ernst, Bronshoj (DK); Carsten Andersen, Vaerlose (DK); Ninna Willestofte Berg, Ballerup (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 12/831,450

(22) Filed: Jul. 7, 2010

Related U.S. Application Data
(63) Continuation of application No. 10/516,164, filed on Nov. 30, 2004.
(60) Provisional application No. 60/393,345, filed on Jul. 1, 2002.

(30) Foreign Application Priority Data
Jun. 26, 2002 (DK) PA 2002 00985

(51) Int. Cl. C12N 9/54

(52) U.S. Cl. 435/221

(57) ABSTRACT

The present invention relates to subtilase variants and subtilases with an altered immunogenicity, particularly subtilase variants and subtilases with a reduced allergenecity. Furthermore, the invention relates to expression of said subtilase variants and subtilases and to their use, such as in detergents and oral care products.

### CROSS-REFERENCE TO RELATED APPLICATIONS

This application is a continuation of U.S. application Ser. No. 10/516,164 filed on Nov. 30, 2004 which is a 35 U.S.C. 371 national application of PCT/DK2003/00434 filed Jun. 25, 2003, which claims priority or the benefit under 35 U.S.C. 119 of Danish application no. PA 2002 00985 filed Jun. 26, 2002 and U.S. provisional application No. 60/393,345 filed Jul. 1, 2002, the contents of which are fully incorporated herein by reference.

### FIELD OF INVENTION

The present invention relates to subtilases and subtilase variants having altered immunogenicity, to the use thereof, as well as to a method for producing said subtilases and subtilase variants.

### BACKGROUND OF THE INVENTION

An increasing number of proteins, including enzymes, are being produced industrially, for use in various industries, housekeeping and medicine. Being proteins they are likely to stimulate an immunological response in man and animals, e.g. an allergic response.

Various attempts to alter the immunogenicity of proteins have been conducted. In general it is only localized parts of the protein, known as epitopes, which are responsible for induction of an immunologic response. An epitope consist of a number of amino acids, which may in the primary sequence be sequential but which more often are located in proximity of each other in the 3-dimensional structure of the protein. It has been found that small changes in an epitope may affect the binding to an antibody. This may result in a reduced importance of such an epitope, maybe converting it from a high affinity to a low affinity epitope, or maybe even result in epitope loss, i.e. that the epitope cannot sufficiently bind an antibody to elicit an immunogenic response.

Another method for altering the immunogenicity of a protein is by “masking the epitopes by e.g. adding compounds, such as PEG, to the protein.

WO 00/26230 and WO 01/83559 disclose two different methods of selecting a protein variant having reduced immunogenicity as compared to the parent protein.

WO 99/38978 discloses a method for modifying allergens to be less allergenic by modifying the IgE binding sites.

WO 99/53038 discloses mutant proteins having lower allergenic response in humans and methods for constructing, identifying and producing such proteins.

Subtilases, which have a wide-spread use within the detergent industry, is a group of enzymes which potentially may elicit an immunogenic response, such as allergy. Thus there is a constant need for subtilases or subtilase variants which have an altered immunogenicity, particularly a reduced allergenicity and which at the same still maintain the enzymatic activity necessary for their application.

WO 00/22103 discloses polypeptides with reduced immune response and WO 01/83559 discloses protein variants having modified immunogenicity.

### BRIEF DESCRIPTION OF THE INVENTION

In a first aspect the present invention relates to a subtilase variant, wherein position 57 is modified in combination with a modification in at least one of the positions: 170, 181, and 247.

In a second aspect the present invention relates to a subtilase of SEQ ID NO. 1, wherein the Xaa residue in position 3 is S or T, in position 4 is V or I, in position 27 is K or R, in position 55 is G, A, V, L, I, T, C, M, P, D, N, E, Q, K, R, H, F, Y, W or absent, in position 74 is N or D, in position 85 is S or N, in position 97 is S or D, in position 99 is S, G or R, in position 101 is S or A, in position 102 is V, N, Y or I, in position 121 is N or S, in position 157 is G, D or S, in position 188 is A or P, in position 193 is V or M, in position 199 is V or I, in position 211 is L or D, in position 216 is M or S, in position 226 is A or V, in position 230 is Q or H, in position 239 is Q or R, in position 242 is N or D, in position 246 is N or K, in position 268 is T or A, and wherein the Xaa residues in positions 164, 175 and 241 are one of the following combinations
a) the Xaa in position 164 is G, A, V, L, I, S, T, C, M, P, D, N, E, Q, K, H, F, Y, W or absent, the Xaa in position 175 is G, A, V, L, I, S, T, C, M, P, D, N, E, Q, K, R, H, F, Y, W or absent and the Xaa in position 241 is G, A, V, L, I, S, T, C, M, P, D, N, E, Q, K, R, H, F, Y, W, or absent or
b) the Xaa in position 164 is G, A, V, L, I, S, T, C, M, P, D, N, E, Q, K, R, H, F, Y, W or absent, the Xaa in position 175 is G, A, V, L, I, S, T, C, M, P, N, E, Q, K, R, H, F, Y, W or absent and the Xaa in position 241 is G, A, V, L, I, S, T, C, M, P, D, N, E, Q, K, R, H, F, Y, W or absent or
c) the Xaa in position 164 is G, A, V, L, I, S, T, C, M, P, D, N, E, Q, K, R, H, F, Y, W or absent, the Xaa in position 175 is G, A, V, L, I, S, T, C, M, P, D, N, E, Q, K, R, H, F, Y, W or absent and the Xaa in position 241 is G, A, V, L, I, S, T, C, M, P, D, N, E, Q, K, H, F, Y, W or absent.

In a third aspect the present invention relates to a DNA sequence encoding a subtilase and/or a subtilase variant of the present invention.

In a fourth aspect the present invention relates to a vector comprising said DNA sequence.

In a fifth aspect the present invention relates to a host cell comprising said vector.

In a sixth aspect the present invention relates to a composition comprising a subtilase and/or a subtilase variant of the present invention.

### Definitions

The term “subtilase” is in the context of the present invention to be understood as a sub-group of serine proteases as described by Siezen et al., Protein Engng. 4 (1991) 719-737 and Siezen et al. Protein Science 6 (1997) 501-523.

The term “parent” is in the context of the present invention to be understood as a protein, which is modified to create a protein variant. The parent protein may be a naturally occurring (wild-type) polypeptide or it may be a variant thereof prepared by any suitable means. For instance, the parent protein may be a variant of a naturally occurring protein which has been modified by substitution, chemical modification, deletion or truncation of one or more amino acid residues, or by addition or insertion of one or more amino acid residues to the amino acid sequence, of a naturally-occurring polypeptide. Thus the term “parent subtilase” refers to a subtilase which is modified to create a subtilase variant.

The term “variant” is in the context of the present invention to be understood as a protein which has been modified as compared to a parent protein at one or more amino acid residues.

The term “modification(s)” or “modified” is in the context of the present invention to be understood as to include chemical modification of a protein as well as genetic manipulation of the DNA encoding a protein. The modification(s) may be replacement(s) of the amino acid side chain(s), substitution(s), deletion(s) and/or insertions in or at the amino acid(s) of interest. Thus the term “modified protein”, e.g. “modified subtilase”, is to be understood as a protein which contains modification(s) compared to a parent protein.

The term “position” is in the present invention to be understood as the number from the N-terminal end of an amino acid in a protein. The position numbers used in the present invention refer to the positions of Subtilisin Novo (BPN′) (SEQ ID NO:2) from B. amyloliquefaciens. However, other subtilases are also covered by the present invention. The corresponding positions of other subtilases are defined by alignment with Subtilisin Novo (BPN′) (SEQ ID NO:2) from B. amyloliquefaciens by using the GAP program. GAP is provided in the GCG program package (Program Manual for the Wisconsin Package, Version 8, August 1994, Genetics Computer Group, 575 Science Drive, Madison, Wis., USA 53711) (Needleman, S. B. and Wunsch, C. D., (1970), Journal of Molecular Biology, 48, 443-45). Unless specified, positions mentioned in the present invention, are given in the BPN′ numeration, and can be converted by alignment.

The term “protein” is in the context of the present invention intended to cover oligopeptides, polypeptides as well as proteins as such.

The term “deletion” or “deleted”, used in relation to a position or an amino acid, refers in the context of the present invention to that the amino acid in the particular position has been deleted or that it is absent.

The term “insertion” or “inserted”, used in relation to a position or amino acid, refers in the context of the present invention to that 1 or more amino acids, e.g. between 1-5 amino acids, have been inserted or that 1 or more amino acids, e.g. between 1-5 amino acids are present after the amino acid in the particular position

The term “substitution” or “substituted”, used in relation to a position or amino acid, refers in the context of the present invention to that the amino acid in the particular position has been replaced by another amino acid or that an amino acid different from the one of a specified protein, e.g. protein sequence, is present.

### Abbreviations

### SEQ ID NO.1′:

The term SEQ ID NO.1′ is in the context of the present invention used as an abbreviation for a sequence according to SEQ ID NO.1, wherein the Xaa residue
in position 3 is S or T,
in position 4 is V or I,
in position 27 is K or R,
in position 55 is G, A, V, L, I, T, C, M, P, D, N, E, Q, K, R, H, F, Y, W or absent
in position 74 is N or D,
in position 85 is S or N,
in position 97 is S or D,
in position 99 is S, G or R,
in position 101 is S or A,
in position 102 is V, N, Y or I,
in position 121 N or S,
in position 157 is G, D or S,
in position 188 is A or P,
in position 193 is V or M,
in position 199 is V or I,
in position 211 is L or D,
in position 216 is M or S,
in position 226 is A or V,
in position 230 is Q or H,
in position 239 is Q or R,
in position 242 is N or D,
in position 246 is N or K,
in position 268 is T or A,
   and wherein the Xaa residues in positions 164, 175 and 241 are one of the following combinations:
a) the Xaa in position 164 is G, A, V, L, I, S, T, C, M, P, D, N, E, Q, K, H, F, Y, W or absent, the Xaa in position 175 is G, A, V, L, I, S, T, C, M, P, D, N, E, Q, K, R, H, F, Y, W or absent and the Xaa in position 241 is G, A, V, L, I, S, T, C, M, P, D, N, E, Q, K, R, H, F, Y, W, or absent or
b) the Xaa in position 164 is G, A, V, L, I, S, T, C, M, P, D, N, E, Q, K, R, H, F, Y, W or absent, the Xaa in position 175 is G, A, V, L, I, S, T, C, M, P, N, E, Q, K, R, H, F, Y, W or absent and the Xaa in position 241 is G, A, V, L, I, S, T, C, M, P, D, N, E, Q, K, R, H, F, Y, W or absent or
c) the Xaa in position 164 is G, A, V, L, I, S, T, C, M, P, D, N, E, Q, K, R, H, F, Y, W or absent, the Xaa in position 175 is G, A, V, L, I, S, T, C, M, P, D, N, E, Q, K, R, H, F, Y, W or absent and the Xaa in position 241 is G, A, V, L, I, S, T, C, M, P, D, N, E, Q, K, H, F, Y, W or absent.

### Amino Acids

The well-known three-letter and one-letter abbreviations for amino acids is used (see e.g. Creighton T E (1993), Proteins; Structures and Molecular Properties, 2^{nd} Edition W.H: Freeman and Company, FIG. 1.1, p. 3). The abbreviation “X” or “Xaa” is used for any amino acid. Within the context of the present invention the abbreviation “aa” is used for “amino acid”.

### Variants

To describe a deletion, an insertions and/or a substitution of amino acid(s) the following nomenclature is used in the present invention.
Original amino acid(s), position(s), deleted/inserted/substituted amino acid(s)
According to this the substitution of Glutamic acid for glycine in position 195 is designated as:
Gly 195 Glu or G195E
   a deletion of glycine in the same position is:
Gly 195* or G195*
   and insertion of an additional amino acid residue such as lysine is:
Gly 195 GlyLys or G195GK
   Where a deletion in comparison with the sequence used for the numbering is indicated, an insertion in such a position is indicated as:
*36 Asp or *36D
   for insertion of an aspartic acid in position 36
   Multiple mutations are separated by pluses, i.e.:

Arg 170 Tyr+Gly 195 Glu or R170Y+G195E

representing mutations in positions 170 and 195 substituting tyrosine and glutamic acid for arginine and glycine, respectively.

### DETAILED DESCRIPTION OF THE INVENTION

### Subtilase Variants and Subtilases of the Invention

The present invention relates to subtilase variants, wherein position 57 is modified in combination with a modification in at least one of the positions: 170, 181, and 247 and to subtilases of SEQ ID NO.1′. The inventors have found that said subtilase variants and subtilases have an altered immunogenicity in comparison to the parent subtilase and Savinase, respectively.

The amino acids in positions 57, 170, 181 and/or 247 of a subtilase variant of the present invention may be modified by genetic manipulation of the DNA encoding the parent subtilase or by chemical modification of for example amino acid side chain(s). In particular said positions may be modified by genetic manipulation of the DNA encoding the parent subtilase, e.g. by deletion, insertion or substitution. An insertion may typically involve inserting between 1 to 5 amino acids, such as 1, 2, 3, 4 or 5 amino acids.

In a particular embodiment of the invention positions 57, 170, 181 and/or 247 in a subtilase variant of the present invention may be modified by substitution. Particularly, substitution of the amino acid in position 57, 170, 181 and/or 247 may involve substitution to an amino acid of different size, hydrophilicity, and/or polarity, such as a small amino acid versus a large amino acid, a hydrophilic amino acid versus a hydrophobic amino acid, a polar amino acid versus a non-polar amino acid and a basic versus an acidic amino acid as these types of substitutions often alter the immunogenicity. The substitution may also involve substitution to an amino acid suitable for chemical modification, such as substitution to a Lysine (K), Aspartic acid (D), Glutamic acid (E) or Cysteine (C). More particularly the amino acid (aa) residue in position 57 may be substituted to one of the residues: P, K, L, A, W, R, H, C, D, I, the aa residue in position 170 may be modified to one of the residues: C, F, G, I, M, N, P, Q, S, T, V, W, Y, A, L, E, D, K, H, the aa residue in position 181 may be modified to one of the residues: A, C, F, G, H, I, K, L, M, N, P, Q, R, S, T, V, Y, E, W and/or the aa residue in position 247 may be modified to one of the residues: A, C, D, E, G, H, I, K, L, M, N, P, Q, S, T, V, F, Y.

For example the subtilase variant of the present invention may be X57P, K, L, A, W, R, H, C, D, I+X170C, F, G, I, M, N, P, Q, S, T, V, W, Y, A, L, E, D, K, H, or it may be X57P, K, L, A, W, R, H, C, D, I+X181A, C, F, G, H, I, K, L, M, N, P, Q, R, S, T, V, Y, E, W or it may be X57P, K, L, A, W, R, H, C, D, I+X247A, C, D, E, G, H, I, K, L, M, N, P, Q, S, T, V, F, Y or it may be X57P, K, L, A, W, R, H, C, D, I+X170C, F, G, I, M, N, P, Q, S, T, V, W, Y, A, L, E, D, K, H+X247A, C, D, E, G, H, I, K, L, M, N, P, Q, S, T, V, F, Y or it may be X57P, K, L, A, W, R, H, C, D, I+X181A, C, F, G, H, I, K, L, M, N, P, Q, R, S, T, V, Y, E, W+X247A, C, D, E, G, H, I, K, L, M, N, P, Q, S, T, V, F, Y.

In particular the subtilase variant of the present invention may be one of the following: X57P+X170F, X57P+X170L, X57P+X181N, X57P+X247E, X57P+X247H, X57P+X247K, X57P+X247Q, X57P+X170F+X247E, X57P+X170F+X247H, X57P+X170F+X247K, X57P+X170F+X247Q, X57P+X170L+X247E, X57P+X170L+X247H, X57P+X170L+X247K, X57P+X170L+X247Q, X57P+X181N+X247E, X57P+X181N+X247H, X57P+X181N+X247K, X57P+X181N+X247Q.X57P+X170L, more particularly X57P+X170L+X247Q.

In a particular embodiment the subtilase variant of the present invention may further comprise a substitution, insertion or deletion in one of the positions: 1, 3, 4, 27, 36, 76, 87, 97, 98, 99, 100, 101, 103, 104, 120, 123, 159, 160, 166, 167, 169, 170, 194, 195, 199, 205, 217, 218, 222, 232, 235, 236, 245, 248, 252, 274. Particularly, those modifications may be one or more of the following: X1 G, X3T, X4I, X27L, X27R, X36*, X76D, X87N, X99D, X101G, X101R, X103A, X104I, X104N, X104Y, X120D, X123S, X159D, X160S, X167A, X170S, X194P, X195E, X199M, X205I, X217D, X217L, X218S, X222S, X222A, X232V, X235L, X236H, X245R, X248D, X252K, X274A.

In another embodiment the subtilase variant of the present invention may further comprise an insertion in a loop, i.e. an insertion in one or more of positions 33-43, 95-103, 125-132, 153-173, 181-195, 202-204 or 218-219.

The present invention also relates to a subtilase according to SEQ ID No.1′. In one embodiment of the invention it may be a subtilase according to SEQ ID NO.1′, wherein the Xaa in position 55, 164, 175 and/or 241 are deleted or comprise an insertion, such as an insertion of between 1-5 amino acids, e.g. an insertion of 1, 2, 3, 4 or 5 amino acids. The Xaa in position 55, 164, 175 and/or 241 may also be an amino acid suitable for chemical modification, such as Lysine (K), Aspartic acid (D), Glutamic acid (E) or Cysteine (C).

In another embodiment the Xaa in position 55 may be one of the residues: G, A, V, L, I, T, C, M, P, D, N, E, Q, K, R, H, F, Y, W and/or Xaa in position 164 may be one of the residues C, F, G, I, M, N, P, Q, S, T, V, W, Y, A, L, E, D, K, H and/or Xaa in position 175 may be one of the residues: A, C, F, G, H, I, K, L, M, N, P, Q, R, S, T, V, Y, E, W and/or Xaa in position 241 may be one of the residues: A, C, D, E, G, H, I, K, L, M, N, P, Q, S, T, V, F, Y. Particularly, the Xaa in position 55 may be one of the residues: P, K, L, A, W, R, H, C, D, I

For example the Xaa in position 55 may be one of the residues: P, K, L, A, W, R, H, C, D, I and the Xaa in position 164 may be one of the residues: C, F, G, I, M, N, P, Q, S, T, V, W, Y, A, L, E, D, K, H, or the Xaa in position 55 may be one of the residues: P, K, L, A, W, R, H, C, D, I and the Xaa in position 175 may be one of the residues: A, C, F, G, H, I, K, L, M, N, P, Q, R, S, T, V, Y, E, W, or the Xaa in position 55 may be one of the residues: P, K, L, A, W, R, H, C, D, I and the Xaa in position 241 may be one of the residues: A, C, D, E, G, H, I, K, L, M, N, P, Q, S, T, V, F, Y.

More particularly, the Xaa in position 55 may be one of the residues: P, K, L, A, W, R, H, C, D, I and the Xaa in position 164 may be one of the residues: C, F, G, I, M, N, P, Q, S, T, V, W, Y, A, L, E, D, K, H and the Xaa in position 241 may be one of the residues: A, C, D, E, G, H, I, K, L, M, N, P, Q, S, T, V, F, Y, or the Xaa in position 55 may be one of the residues: P, K, L, A, W, R, H, C, D, I and the Xaa in position 175 may be one of the residues: A, C, F, G, H, I, K, L, M, N, P, Q, R, S, T, V, Y, E, W and the Xaa in position 241 may be one of the residues: A, C, D, E, G, H, I, K, L, M, N, P, Q, S, T, V, F, Y.

The subtilase of the present invention may also be a subtilase according to SEQ ID NO.1′, wherein the combination of Xaa's in position 3, 4, 27, 74, 85, 97, 99, 101, 102, 121, 157, 188, 193, 199, 211, 216, 226, 230, 239, 242, 246 and 268 may be one of the following:
i) in position 3 is S, in position 4 is V, in position 27 is K, in position 74 is N, in position 85 is S, in position 97 is S, in position 99 is S, in position 101 is S, in position 102 is V, in position 121 is N, in position 157 is G, in position 188 is A, in position 193 is V, in position 199 is V, in position 211 is L, in position 216 is M, in position 226 is A, in position 230 is Q, in position 239 is Q, in position 242 is N, in position 246 is N, in position 268 is T
   or
ii) in position 3 is S, in position 4 is V, in position 27 is K, in position 74 is N, in position 85 is N, in position 97 is S, in position 99 is G, in position 101 is S, in position 102 is N, in position 121 is N, in position 157 is G, in position 188 is A, in position 193 is V, in position 199 is V, in position 211 is L, in position 216 is M, in position 226 is A, in position 230 is Q, in position 239 is Q, in position 242 is N, in position 246 is N, in position 268 is T
   or
iii) in position 3 is S, in position 4 is V, in position 27 is K, in position 74 is N, in position 85 is N, in position 97 is S, in position 99 is S, in position 101 is S, in position 102 is V, in position 121 is N, in position 157 is G, in position 188 is A, in position 193 is V, in position 199 is V, in position 211 is L, in position 216 is S, in position 226 is A, in position 230 is Q, in position 239 is Q, in position 242 is N, in position 246 is N, in position 268 is T
   or
iv) in position 3 is S, in position 4 is V, in position 27 is R, in position 74 is N, in position 85 is S, in position 97 is S, in position 99 is S, in position 101 is S, in position 102 is Y, in position 121 is S, in position 157 is G, in position 188 is A, in position 193 is V, in position 199 is V, in position 211 is L, in position 216 is M, in position 226 is A, in position 230 is Q, in position 239 is Q, in position 242 is N, in position 246 is N, in position 268 is A
   or
v) in position 3 is S, in position 4 is V, in position 27 is K, in position 74 is D, in position 85 is S, in position 97 is S, in position 99 is S, in position 101 is A, in position 102 is I, in position 121 is N, in position 157 is G, in position 188 is A, in position 193 is V, in position 199 is V, in position 211 is L, in position 216 is M, in position 226 is A, in position 230 is Q, in position 239 is Q, in position 242 is N, in position 246 is N, in position 268 is T
   or
vi) in position 3 is S, in position 4 is V, in position 27 is K, in position 74 is N, in position 85 is S, in position 97 is S, in position 99 is G, in position 101 is A, in position 102 is I, in position 121 is N, in position 157 is D, in position 188 is A, in position 193 is V, in position 199 is V, in position 211 is L, in position 216 is M, in position 226 is V, in position 230 is H, in position 239 is R, in position 242 is D, in position 246 is K, in position 268 is T
   or
vii) in position 3 is S, in position 4 is V, in position 27 is K, in position 74 is N, in position 85 is S, in position 97 is D, in position 99 is R, in position 101 is A, in position 102 is I, in position 121 is N, in position 157 is S, in position 188 is A, in position 193 is V, in position 199 is V, in position 211 is L, in position 216 is S, in position 226 is A, in position 230 is Q, in position 239 is Q, in position 242 is N, in position 246 is N, in position 268 is T
   or
viii) in position 3 is T, in position 4 is I, in position 27 is K, in position 74 is N, in position 85 is S, in position 97 is S, in position 99 is S, in position 101 is S, in position 102 is V, in position 121 is N, in position 157 is G, in position 188 is P, in position 193 is M, in position 199 is I, in position 211 is D, in position 216 is M, in position 226 is A, in position 230 is Q, in position 239 is Q, in position 242 is N, in position 246 is N, in position 268 is T
   or
ix) in position 3 is T, in position 4 is I, in position 27 is K, in position 74 is N, in position 85 is S, in position 97 is S, in position 99 is S, in position 101 is S, in position 102 is V, in position 121 is N, in position 157 is G, in position 188 is A, in position 193 is M, in position 199 is I, in position 211 is D, in position 216 is M, in position 226 is A, in position 230 is Q, in position 239 is Q, in position 242 is N, in position 246 is N, in position 268 is T
   or
x) in position 3 is T, in position 4 is I, in position 27 is K, in position 74 is N, in position 85 is S, in position 97 is S, in position 99 is S, in position 101 is S, in position 102 is V, in position 121 is N, in position 157 is G, in position 188 is A, in position 193 is V, in position 199 is I, in position 211 is L, in position 216 is M, in position 226 is A, in position 230 is Q, in position 239 is Q, in position 242 is N, in position 246 is N, in position 268 is T.

The subtilase of the present invention may also comprise a substitution, insertion or deletion in one or more of the following positions: 1, 35, 95, 96, 98, 118, 158, 161, 163, 164, 189, 212 and 229. Examples of such modifications include: X1G, X27L, I35ID, X74D, X118D, A158AS, X161A, X164S, X189E, X212S and X229L.

In one embodiment of the invention the subtilase of the present invention may also comprise an insertion in a loop, i.e. an insertion in one or more of positions 33-42, 93-101, 123-130, 151-167, 175-189, 196-198 or 212-213.

### Subtilase

As described above subtilases constitute a sub-group of serine protease according to Siezen et al., Protein Engng. 4 (1991) 719-737 and Siezen et al. Protein Science 6 (1997) 501-523. Subtilases are defined by homology analysis of more than 170 amino acid sequences of serine proteases previously referred to as subtilisin-like proteases. The subtilases may be divided into 6 sub-divisions, i.e. the Subtilisin family, the Thermitase family, the Proteinase K family, the Lantibiotic peptidase family, the Kexin family and the Pyrolysin family. The Subtilisin family may be further divided into 3 sub-groups, i.e. I-S1 (“true” subtilisins), I-S2 (highly alkaline proteases) and intracellular subtilisins. Definitions or grouping of enzymes may vary or change, however, in the context of the present invention the above division of subtilases into sub-division or sub-groups shall be understood as those described by Siezen et al., Protein Engng. 4 (1991) 719-737 and Siezen et al. Protein Science 6 (1997) 501-523.

The subtilase variants of the present invention are obtained by modification of a parent subtilase.

The parent subtilase and/or the subtilase of the present invention may be a subtilase isolated from natural source, i.e. a wild type subtilase, or it may be a subtilase isolated from a natural source in which subsequent modifications have been made while retaining the characteristic of a subtilase. Examples of such subtilase variants which may be parent subtilases include those disclosed in EP 130.756, EP 214.435, WO 87/04461, WO 87/05050, EP 251.446, EP 260.105, WO 88/08028, WO 88/08033, WO 89/06279, WO 91/00345, EP 525 610 and WO 94/02618. In another embodiment the parent subtilase may be a subtilase which has been prepared by a DNA shuffling technique, such as described by J. E. Ness et al., Nature Biotechnology, 17, 893-896 (1999). Further, a parent subtilase may be constructed by standard techniques for artificial creation of diversity, such as by DNA shuffling of different subtilase genes (WO 95/22625; Stemmer W P C, Nature 370:389-91 (1994)). For example the parent subtilase may be constructed by DNA shuffling of e.g. the gene encoding Savinase® with one or more partial subtilase sequences identified in nature.

In particular the parent subtilase and/or a subtilase of the present invention may be a subtilisin, more particular a subtilisin belonging to the I-S1 or the I-S2 group. Examples I-S1 subtilases include subtilisin BPN′, subtilisin amylosaccharitus, subtilisin 168, subtilisin mesentericopeptidase, subtilisin Carlsberg (Alcalase®)and subtilisin DY. Examples of I-S2 subtilases include subtilisin 309 (Savinase), subtilisin 147, subtilisin PB92, BLAP and K16.

In another embodiment the parent subtilase and/or the subtilase of the present invention may be a subtilase belonging to the Thermitase family, e.g. Thermitase.

The parent subtilase and/or a subtilase of the present invention may also belong to the Proteinase K family, such as Proteinase K.

Other examples of subtilases which may be used as parent subtilases include PD498 (WO 93/24623), aqualysin, protease TW7, protease TW3, high-alkaline proteases such as those described in EP503346, EP610808 and WO 95/27049.

In another embodiment the parent subtilase may be subtilase in which subsequent modifications have been made while retaining the characteristic of a subtilase. For example the parent subtilase may comprise an insertion in a loop, i.e. an insertion in one or more of positions 33-43, 95-103, 125-132,153-173, 181-195, 202-204 or 218-219. The parent subtilase may also be Savinase in which further modifications have been made. Examples of such further modifications include a substitution, insertion or deletion in one or more of the following positions: 1, 3, 4, 27, 36, 76, 87, 97, 98, 99, 100, 101, 103, 104, 120, 123, 159, 160, 166, 167, 169, 170, 194, 195, 199, 205, 217, 218, 222, 232, 235, 236, 245, 248, 252, 274. Examples of such modifications include: X1G, X3T, X4I, X27L, S27R, *36D, X76D, X87N, X99D, X101G, X101R, X103A, X104I, X104N, X104Y, X120D, X123S, X159D, X160S, X167A, X170S, X194P, X195E, X199M, X205I, X217D, X217L, X218S, X222S, X222A, X232V, X235L, X236H, X245R, X248D, X252K, X274A.

In particular the parent subtilase and/or subtilase of the present invention may a Savinase-like subtilisins, i.e. having at least 40% identity to Savinase, such as at least 50% identity or at least 60% identity, more particularly at least 70% identity or at least 80% identity, even more particularly at least 90% identity or at least 95% identity to Savinase, wherein the identity is between the nucleic acid sequence of the parent subtilase/the subtilase of the present invention respectively, compared to the nucleic acid sequence of Savinase.

Alignment of various subtilisin proteases to Savinase reveal that the identity between the nucleic acid sequences of various subtilisin proteases ranges between 100% and 40%.

Sequence identities between different pairs of proteases are given below:

### Sequence Identity to Savinase:

| | |
|---|---|
| Alcalase ® | 60.9% |
| BLAPR | 98.1% |
| ProteaseC | 98.5% |
| ProteaseD | 98.9% |
| ProteaseE | 96.7% |
| ProteaseA | 97.8% |
| Properase ™ | 98.9% |
| Relase ® | 98.1% |
| PD498 | 44.3% |
| sendai | 81.4% |
| YAB | 81.8% |

The protein structure of PD498 is disclosed in WO98/35026 (Novo Nordisk). The structure of Savinase can be found in BETZEL et al, J. MOL. BIOL., Vol. 223, p. 427, 1992 (1svn.pdb).

The activity of subtilases and subtilase variants can be determined as described in “Methods of Enzymatic Analysis”, third edition, 1984, Verlag Chemie, Weinheim, vol. 5.

### Immunogenicity

The inventors of the present invention have found that the subtilase variant and subtilases of the present invention have an altered immunogenicity as compared to the parent subtilase and to Savinase, respectively.

An “immunological response” is in the present invention to be understood as the response of an organism to a compound, which involves the immune system according to any of the four standard reactions (Type I, II, Ill and IV according to Coombs & Gell). Correspondingly, the term “immunogenicity” of a compound used in connection with the present invention refers to the ability of this compound to induce an immunological response in animals including man.

The term “altered immunogenicity” when used in relation to a subtilase variant or subtilase of the present invention refers to that an immunologic response of an organism to said subtilase variant/subtilase is different, i.e. decreased or increased, compared to the same type of immunologic response to the parent subtilase/Savinase, respectively.

Typically it is only parts of the protein, also called epitopes, which are involved in induction of an immunologic response, such as antibody binding or T-cell activation. Typically the epitopes consist of a set of non-sequential amino acids, i.e. amino acids which are not located next to each other in the primary sequence but which in the 3-dimensional structure of the protein are located in proximity of each other. One particularly useful method of identifying epitopes involved in antibody binding is to screen a library of peptide-phage membrane protein fusions and selecting those that bind to relevant antigen-specific antibodies, sequencing the randomized part of the fusion gene, aligning the sequences involved in binding, defining consensus sequences based on these alignments, and mapping these consensus sequences on the surface or the sequence and/or structure of the antigen, to identify epitopes involved in antibody binding. Methods of identifying epitopes are described in WO 01/83559 and WO 99/53038.

Allergy is in general understood as an adverse immunologic response to an innocuous foreign substance due to the presence of pre-existing antibodies and T-cells (Janeway and Travers, Immunology, Current Biology, Blackwell, Garland, 1994, chapter 11). Most allergic responses involve an IgE mediated response and in the context of the present invention the term “allergic response” is to be understood as the response of an organism to a compound, which involves IgE mediated responses (Type I reaction according to Coombs & Gell). It is to be understood that sensibilization (i.e. development of compound-specific IgE antibodies) upon exposure to the compound is included in the definition of “allergic response”. Correspondingly, the term “allergenicity” of a compound used in connection with the present invention refers to the ability of this compound to induce an allergic response in animals including man.

The general mechanism behind an allergic response is divided in a sensitisation phase and a symptomatic phase. The sensitisation phase involves a first exposure of an individual to an allergen, which depending on the application may occur by inhalation, direct contact with the skin and eyes, or injection. This event activates specific T- and B-lymphocytes, and leads to the production of allergen specific IgE antibodies, i.e. immunoglobulin E. These IgE antibodies eventually facilitate allergen capturing and presentation to T-lymphocytes at the onset of the symptomatic phase. This phase is initiated by a second exposure to the same or a resembling antigen. The specific IgE antibodies bind to specific IgE receptors on mast cells and basophiles, among others, and capture at the same time the allergen. As the IgE antibodies are polyclonal the result is bridging and clustering of the IgE receptors, which activate the mast cells and basophiles. This activation triggers the release of various chemical mediators involved in the early as well as late phase reactions of the symptomatic phase of allergy.

In particular the subtilase variants and for subtilases of the present invention may have a reduced immunogenicity, such as a reduced allergenicity.

Allergenicity should in the context of the present invention be measured as the IgE response generated in Balb/C mice by subcutaneous immunisisation of the mice weekly, for a period of 20 weeks, with 50 microl 0.9% (wt/vol) NaCl (control group), or 50 microl 0.9% (wt/vol) NaCI containing 10 microg of protein, collecting serum from the eye every other week before the next immunization and then determining the IgE levels using an ELISA specific for mouse IgE.

Thus the term reduced allergenicity used in connection with the subtilases variants/subtilases of the present invention is to be understood as an IgE response which is less or none in said assay compared to the parent subtilase/Savinase, respectively. In particular the IgE level measured in said assay obtained in response to said subtilase variants and/or subtilases may be 35%, such as 30% or 25% or 20% or 15% or 10% of the IgE level obtained in response to the parent subtilase/Savinase, respectively. Thus the IgE response to the subtilase variants and/or subtilases of the present invention may be reduced at least 3 times, such as 5 times or 10 times compared to the parent subtilase/Savinase, respectively.

Other methods which may be used for testing for an immunologic/allergic response to a protein include in vitro assays, such as assays testing the antibody binding and/or functionality of the protein which may be examined in detail using dose-response curves and e.g. direct or competitive ELISA (C-ELISA), such as described in (WO 99/47680), assays based on cytokine expression profiles and assays based on proliferation or differentiation responses of epithelial and other cells incl. B-cells and T-cells. Examples of in vivo models for testing the allergenicity include the guinea pig intratracheal model (GPIT) (Ritz, et al. Fund. Appl.Toxicol., 21, pp. 31-37, 1993), mouse subcutaneous model (mouse-SC) (WO 98/30682), the rat intratracheal model (rat-IT) (WO 96/17929) and the mouse intranasal model (MINT) (Robinson et al., Fund. Appl. Toxicol., 34, pp. 15-24, 1996).

The subtilase variants and/or subtilases of the present invention may be tested for altered allergenicity and/or immunogenicity by using a purified preparation of the subtilase variants/subtilases, respectively. Thus before testing the subtilase variants or subtilases for altered allergenicity and/or immunogenicity they may be expressed in larger scale and/or purified by conventional methods.

### Further Modifications

The subtilase variants and/or subtilases of the present invention may be further modified by e.g. mutations and/or chemical conjugation. The purpose of this may be to decrease the allergenicity further or to increase the performance, the stability, the thermostability or any other feature of the enzyme.

In one embodiment of the invention the subtilase variants and/or subtilases may be further modified by substitutions in the protein for example so that amino acids suitable for chemical modification are substituted for existing ones within, for example in epitope areas. Particularly, the substitutions may be conservative to limit the impact on the protein structure, for example the substitution may be arginine to lysine, asparagine to aspartic acid, glutamine to glutamic acid, threonine or serine to cysteine. Chemical modification may also be performed on amino acids present in the subtilase variants and/or subtilases of the present invention without first substituting one or more amino acids with other amino acids. The chemistry for chemical modification is described above.

In a particular embodiment of the invention the subtilase variants and/or subtilases of the present invention may be further modified to further reduce the allergenicity of said enzymes. In particular the subtilase variants and/or subtilases of the present invention may be further modified by the method described in WO 99/00489, wherein polymeric molecules having a molecular weight from 100 Da to below 750 Da, particularly from 100 to 500 Da, such as around 300 Da are coupled to the protein. The polymeric molecules may be any suitable polymeric molecule including natural and synthetic homo-polymers, such as polyols (i.e. poly-OH), polyamines (i.e. poly-NH2) and polycarboxyl acids (i.e. poly-COOH), and further hetero-polymers i.e. polymers comprising one or more different coupling groups e.g. a hydroxyl group and amine groups. Specific examples include polyethylene glycols (PEG), methoxypolyethylene glycols (mPEG) and polypropylen glycols. The polymers may be coupled to the subtilase variants and/or subtilases by any method known to the person skilled in the art. Typically, 4 to 50 polymeric molecules, such as 5 to 35 polymeric molecules may be coupled to the said enzymes.

Other means for further modifying the subtilase variants/subtilases of the present invention include introduction of recognition sites for post-translational modifications in, e.g. epitope areas of the subtilase variants/subtilases. The subtilase variants/subtilases should then be expressed in a suitable host organism capable of the corresponding post-translational modification. These post-translational modifications may serve to shield the epitope and hence lower the allergenicity and/or immunogenicity of the subtilase variants/subtilases compared to the parent subtilase/Savinase respectively, further. Post-translational modifications include glycosylation, phosphorylation, N-terminal processing, acylation, ribosylation and sulfatation. A good example is N-glycosylation. N-glycosylation is found at sites of the sequence Asn-Xaa-Ser, Asn-Xaa-Thr, or Asn-Xaa-Cys, in which neither the Xaa residue nor the amino acid following the tri-peptide consensus sequence is a proline (T. E. Creighton, ‘Proteins—Structures and Molecular Properties, 2nd edition, W.H. Freeman and Co., New York, 1993, pp. 91-93). The specific nature of the glycosyl chain of the glycosylated protein variant may be linear or branched depending on the protein and the host cells. Another example is phosphorylation: The protein sequence can be modified so as to introduce serine phosphorylation sites with the recognition sequence arg-arg-(xaa)n-ser (where n=0, 1, or 2), which can be phosphorylated by the cAMP-dependent kinase or tyrosine phosphorylation sites with the recognition sequence—lys/arg-(xaa)3-asp/glu-(xaa)3-tyr, which can usually be phosphorylated by tyrosine-specific kinases (T. E. Creighton, “Proteins—Structures and molecular proper-ties”, 2nd ed., Freeman, NY, 1993).

### Chemical Modifications

The subtilase variants and/or subtilases of the present invention may be chemically modified. Any method known to person skilled in the art may be used to chemically modify said enzymes.

The chemistry for preparation of covalent bioconjugates can be found in “Bioconjugate Techniques”, Hermanson, G. T. (1996), Academic Press Inc.

If the subtilase variants are modified by substitution of the amino acids in position 57, 170, 181 and/241 to amino acids which are suitable for chemical modification the substitution may particularly be conservative to secure that the impact of the substitution on the polypeptide structure is limited. In the case of providing additional amino groups this may be done by substitution of arginine to lysine, both residues are positively charged, but only the lysine having a free amino group suitable as an attachment groups. In the case of providing additional carboxylic acid groups the conservative substitution may for instance be an asparagine to aspartic acid or glutamine to glutamic acid substitution. These residues resemble each other in size and shape, except from the carboxylic groups being present on the acidic residues. In the case of providing SH-groups the conservative substitution may be done by changing threonine or serine to cysteine.

### Chemical Conjugation

For chemical conjugation, the protein needs to incubate with an active or activated polymer and subsequently separated from the unreacted polymer. This can be done in solution followed by purification or it can conveniently be done using the immobilized proteins, which can easily be exposed to different reaction environments and washes.

In the case were polymeric molecules are to be conjugated with the polypeptide in question and the polymeric molecules are not active they must be activated by the use of a suitable technique. It is also contemplated according to the invention to couple the polymeric molecules to the polypeptide through a linker. Suitable linkers are well-known to the skilled person. Methods and chemistry for activation of polymeric molecules as well as for conjugation of polypeptides are intensively described in the literature. Commonly used methods for activation of insoluble polymers include activation of functional groups with cyanogen bromide, periodate, glutaraldehyde, biepoxides, epichlorohydrin, divinylsulfone, carbodiimide, sulfonyl halides, trichlorotriazine etc. (see “Bioconjugate Techniques”, Hermanson, G. T. (1996), Academic Press Inc.; “Protein immobilisation. Fundamental and applications”, R. F. Taylor (1991), Marcel Dekker, N.Y.; “Chemistry of Protein Conjugation and Crosslinking”, S. S. Wong (1992), CRC Press, Boca Raton; “Immobilized Affinity Ligand Techniques”, G. T. Hermanson et al. (1993), Academic Press, N.Y.). Some of the methods concern activation of insoluble polymers but are also applicable to activation of soluble polymers e.g. periodate, trichlorotriazine, sulfonylhalides, divinylsulfone, carbodiimide etc. The functional groups being amino, hydroxyl, thiol, carboxyl, aldehyde or sulfydryl on the polymer and the chosen attachment group on the protein must be considered in choosing the activation and conjugation chemistry which normally consist of i) activation of polymer, ii) conjugation, and iii) blocking of residual active groups.

In the following a number of suitable polymer activation methods will be described shortly. However, it is to be understood that also other methods may be used.

Coupling polymeric molecules to the free acid groups of polypeptides may be performed with the aid of diimide and for example amino-PEG or hydrazino-PEG (Pollak et al., (1976), J. Am. Chem. Soc., 98, 289 291) or diazoacetate/amide (Wong et al., (1992), “Chemistry of Protein Conjugation and Crosslinking”, CRC Press).

Coupling polymeric molecules to hydroxy groups is generally very difficult as it must be performed in water. Usually hydrolysis predominates over reaction with hydroxyl groups.

Coupling polymeric molecules to free sulfhydryl groups can be achieved with special groups like maleimido or the ortho-pyridyl disulfide. Also vinylsulfone (U.S. Pat. No. 5,414,135, (1995), Snow et al.) has a preference for sulfhydryl groups but is not as selective as the other mentioned.

Accessible arginine residues in the polypeptide chain may be targeted by groups comprising two vicinal carbonyl groups.

Techniques involving coupling of electrophilically activated PEGs to the amino groups of lysines may also be useful. Many of the usual leaving groups for alcohols give rise to an amine linkage. For instance, alkyl sulfonates, such as tresylates (Nilsson et al., (1984), Methods in Enzymology vol. 104, Jacoby, W. B., Ed., Academic Press: Orlando, p. 56 66; Nilsson et al., (1987), Methods in Enzymology vol. 135; Mosbach, K., Ed.; Academic Press: Orlando, pp. 65 79; Scouten et al., (1987), Methods in Enzymology vol. 135, Mosbach, K., Ed., Academic Press: Orlando, 1987; pp 79 84; Crossland et al., (1971), J. Amr. Chem. Soc. 1971, 93, pp. 4217 4219), mesylates (Harris, (1985), supra; Harris et al., (1984), J. Polym. Sci. Polym. Chem. Ed. 22, pp 341 352), aryl sulfonates like tosylates, and para-nitrobenzene sulfonates can be used.

Organic sulfonyl chlorides, e.g. Tresyl chloride, effectively converts hydroxy groups in a number of polymers, e.g. PEG, into good leaving groups (sulfonates) that, when reacted with nucleophiles like amino groups in polypeptides allow stable linkages to be formed between polymer and polypeptide. In addition to high conjugation yields, the reaction conditions are in general mild (neutral or slightly alkaline pH, to avoid denaturation and little or no disruption of activity), and satisfy the non-destructive requirements to the polypeptide.

Tosylate is more reactive than the mesylate but also less stable decomposing into PEG, dioxane, and sulfonic acid (Zalipsky, (1995), Bioconjugate Chem., 6, 150 165). Epoxides may also been used for creating amine bonds but are much less reactive than the abovementioned groups.

Converting PEG into a chloroformate with phosgene gives rise to carbamate linkages to Lysines. Essentially the same reaction can be carried out in many variants substituting the chlorine with N-hydroxy succinimide (U.S. Pat. No. 5,122,614, (1992); Zalipsky et al., (1992), Biotechnol. Appl. Biochem., 15, p. 100 114; Mon-fardini et al., (1995), Bioconjugate Chem., 6, 62 69, with imidazole (Allen et al., (1991), Carbohydr. Res., 213, pp 309 319), with paranitrophenol, DMAP (EP 632 082 A1, (1993), Looze, Y.) etc. The derivatives are usually made by reacting the chloroformate with the desired leaving group. All these groups give rise to carbamate linkages to the peptide.

Furthermore, isocyanates and isothiocyanates may be employed, yielding ureas and thioureas, respectively.

Amides may be obtained from PEG acids using the same leaving groups as mentioned above and cyclic imid thrones (U.S. Pat. No. 5,349,001, (1994), Greenwald et al.). The reactivity of these compounds is very high but may make the hydrolysis to fast.

PEG succinate made from reaction with succinic anhydride can also be used. The hereby comprised ester group make the conjugate much more susceptible to hydrolysis (U.S. Pat. No. 5,122,614, (1992), Zalipsky). This group may be activated with N-hydroxy succinimide.

Furthermore, a special linker can be introduced. The most well studied being cyanuric chloride (Abuchowski et al., (1977), J. Biol. Chem., 252, 3578 3581; U.S. Pat. No. 4,179,337, (1979), Davis et al.; Shafer et al., (1986), J. Polym. Sci. Polym. Chem. Ed., 24, 375 378.

Coupling of PEG to an aromatic amine followed by diazotation yields a very reactive diazonium salt, which can be reacted with a peptide in situ. An amide linkage may also be obtained by re-acting an azlactone derivative of PEG (U.S. Pat. No. 5,321,095, (1994), Greenwald, R. B.) thus introducing an additional amide linkage.

As some peptides do not comprise many Lysines it may be advantageous to attach more than one PEG to the same Lysine. This can be done e.g. by the use of 1,3-diamino-2-propanol.

PEGs may also be attached to the amino-groups of the enzyme with carbamate linkages (WO 95/11924, Greenwald et al.). Lysine resi-dues may also be used as the backbone.

The coupling technique used in the examples is the N-succinimidyl carbonate conjugation technique descried in WO 90/13590 (Enzon).

In a particular embodiment, the activated polymer is methyl-PEG which has been activated by N-succinimidyl carbonate as described WO 90/13590. The coupling can be carried out at alkaline conditions in high yields.

For coupling of polymers to proteins, in particular conditions similar to those described in WO96/17929 and WO99/00489 (Novo Nordisk NS), e.g. mono or bis activated PEG's of molecular weight ranging from 100 to 5000 Da, may be used. For instance, a methyl-PEG 350 could be activated with N-succinimidyl carbonate and incubated with protein variant at a molar ratio of more than 5 calculated as equivalents of activated PEG divided by moles of lysines in the protein of interest. For coupling to immobilized protein variant, the PEG:protein ratio should be optimized such that the PEG concentration is low enough for the buffer capacity to maintain alkaline pH throughout the reaction; while the PEG concentration is still high enough to ensure sufficient degree of modification of the protein. Further, it is important that the activated PEG is kept at conditions that prevent hydrolysis (i.e. dissolved in acid or solvents) and diluted directly into the alkaline reaction buffer. It is essential that primary amines are not present other than those occurring in the lysine residues of the protein. This can be secured by washing thoroughly in borate buffer. The reaction is stopped by separating the fluid phase containing unreacted PEG from the solid phase containing protein and derivatized protein. Optionally, the solid phase can then be washed with Tris buffer, to block any unreacted sites on PEG chains that might still be present.

### Methods for Production of Subtilase Variants and Subtilases

The subtilase variants and subtilases of the present invention may be produced by any known method within the art and the present invention also relates to nucleic acid encoding a subtilase variant or subtilase of the present invention, a DNA construct comprising said nucleic acid and a host cell comprising said nuclei acid sequence.

In general natural occurring proteins may be produced by culturing the organism expressing the protein and subsequently purifying the protein or it may be produced by cloning a nucleic acid, e.g. genomic DNA or cDNA, encoding the protein into an expression vector, introducing said expression vector into a host cell, culturing the host cell and purifying the expressed protein.

Typically protein variants may be produced by site-directed mutagenesis of a parent protein, introduction into expression vector, host cell etc. The parent protein may be cloned from a strain producing the polypeptide or from an expression library, i.e. it may be isolated from genomic DNA or prepared from cDNA, or a combination thereof.

In general standard procedures for cloning of genes and/or introducing mutations (random and/or site directed) into said genes may be used in order to obtain a parent subtilase, or subtilase or subtilase variant of the invention. For further description of suitable techniques reference is made to Molecular cloning: A laboratory manual (Sambrook et al. (1989), Cold Spring Harbor lab., Cold Spring Harbor, N.Y.; Ausubel, F. M. et al. (eds.)); Current protocols in Molecular Biology (John Wiley and Sons, 1995; Harwood, C. R., and Cutting, S. M. (eds.)); Molecular Biological Methods for Bacillus (John Wiley and Sons, 1990); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); A Practical Guide To Molecular Cloning (B. Perbal, (1984)) and WO 96/34946.

### Expression Vectors

A recombinant expression vector comprising a nucleic acid sequence encoding a subtilase or subtilase variant of the invention may be any vector that may conveniently be subjected to recombinant DNA procedures and which may bring about the expression of the nucleic acid sequence.

The choice of vector will often depend on the host cell into which it is to be introduced. Examples of a suitable vector include a linear or closed circular plasmid or a virus. The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Examples of bacterial origins of replication are the origins of replication of plasmids pBR322, pUC19, pACYC177, pACYC184, pUB110, pE194, pTA1060, and pAMβ1. Examples of origin of replications for use in a yeast host cell are the 2 micron origin of replication, the combination of CEN6 and ARS4, and the combination of CEN3 and ARS1. The origin of replication may be one having a mutation which makes it function as temperature-sensitive in the host cell (see, e.g., Ehrlich, 1978, Proceedings of the National Academy of Sciences USA 75:1433).

Alternatively, the vector may be one which, when introduced into the host cell, is integrated into the genome and replicated together with the chromosome(s) into which it has been integrated. Vectors which are integrated into the genome of the host cell may contain any nucleic acid sequence enabling integration into the genome, in particular it may contain nucleic acid sequences facilitating integration into the genome by homologous or non-homologous recombination. The vector system may be a single vector, e.g. plasmid or virus, or two or more vectors, e.g. plasmids or virus', which together contain the total DNA to be introduced into the genome of the host cell, or a transposon.

The vector may in particular be an expression vector in which the DNA sequence encoding the subtilase of the invention is operably linked to additional segments or control sequences required for transcription of the DNA. The term, “operably linked” indicates that the segments are arranged so that they function in concert for their intended purposes, e.g. transcription initiates in a promoter and proceeds through the DNA sequence encoding the subtilase variant. Additional segments or control sequences include a promoter, a leader, a polyadenylation sequence, a propeptide sequence, a signal sequence and a transcription terminator. At a minimum the control sequences include a promoter and transcriptional and translational stop signals.

The promoter may be any DNA sequence that shows transcriptional activity in the host cell of choice and may be derived from genes encoding proteins either homologous or heterologous to the host cell.

Examples of suitable promoters for use in bacterial host cells include the promoter of the Bacillus subtilis levansucrase gene (sacB), the Bacillus stearothermophilus maltogenic amylase gene (amyM), the Bacillus licheniformis alpha-amylase gene (amyL), the Bacillus amyloliquefaciens alpha-amylase gene (amyQ), the Bacillus subtilis alkaline protease gene, or the Bacillus pumilus xylosidase gene, the Bacillus amyloliquefaciens BAN amylase gene, the Bacillus licheniformis penicillinase gene (penP), the Bacillus subtilis xylA and xylB genes, and the prokaryotic beta-lactamase gene (Villa-Kamaroff et al., 1978, Proceedings of the National Academy of Sciences USA 75:3727-3731). Other examples include the phage Lambda P_{R} or P_{L} promoters or the E. coli lac, trp or tac promoters or the Streptomyces coelicolor agarase gene (dagA). Further promoters are described in “Useful proteins from recombinant bacteria” in Scientific American, 1980, 242:74-94; and in Sambrook et al., 1989, supra.

Examples of suitable promoters for use in a filamentous fungal host cell are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (as described in U.S. Pat. No. 4,288,627, which is incorporated herein by reference), and hybrids thereof. Particularly preferred promoters for use in filamentous fungal host cells are the TAKA amylase, NA2-tpi (a hybrid of the promoters from the genes encoding Aspergillus niger neutral (-amylase and Aspergillus oryzae triose phosphate isomerase), and glaA promoters. Further suitable promoters for use in filamentous fungus host cells are the ADH3 promoter (McKnight et al., The EMBO J. 4 (1985), 2093-2099) or the tpiA promoter.

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Further useful promoters are obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for yeast host cells are described by Romanos et al., 1992, Yeast 8:423-488. In a mammalian host cell, useful promoters include viral promoters such as those from Simian Virus 40 (SV40), Rous sarcoma virus (RSV), adenovirus, and bovine papilloma virus (BPV).

Examples of suitable promoters for use in mammalian cells are the SV40 promoter (Subramani et al., Mol. Cell Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

The DNA sequence encoding the subtilase or subtilase variant of the invention may also, if necessary, be operably connected to a suitable terminator.

The recombinant vector of the invention may further comprise a DNA sequence enabling the vector to replicate in the host cell in question.

The vector may also comprise a selectable marker, e.g. a gene the product of which complements a defect in the host cell, or a gene encoding resistance to e.g. antibiotics like ampicillin, kanamycin, chloramphenicol, erythromycin, tetracycline, spectinomycine, neomycin, hygromycin, methotrexate, or resistance to heavy metals, virus or herbicides, or which provides for prototrophy or auxotrophs. Examples of bacterial selectable markers are the dal genes from Bacillus subtilis or Bacillus licheniformis, resistance. A frequently used mammalian marker is the dihydrofolate reductase gene (DHFR). Suitable markers for yeast host cells are ADE2, HIS3, LEU2, LYS2, MET3, TRP1, and URA3. A selectable marker for use in a filamentous fungal host cell may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), and glufosinate resistance markers, as well as equivalents from other species. Particularly, for use in an Aspergillus cell are the amdS and pyrG markers of Aspergillus nidulans or Aspergillus oryzae and the bar marker of Streptomyces hygroscopicus. Furthermore, selection may be accomplished by cotransformation, e.g., as described in WO 91/17243, where the selectable marker is on a separate vector.

To direct a subtilase or subtilase variant of the present invention into the secretory pathway of the host cells, a secretory signal sequence (also known as a leader sequence, prepro sequence or pre sequence) may be provided in the recombinant vector. The secretory signal sequence is joined to the DNA sequence encoding the enzyme in the correct reading frame. Secretory signal sequences are commonly positioned 5′ to the DNA sequence encoding the enzyme. The secretory signal sequence may be that normally associated with the enzyme or may be from a gene encoding another secreted protein.

The procedures used to ligate the DNA sequences coding for the present enzyme, the promoter and optionally the terminator and/or secretory signal sequence, respectively, or to assemble these sequences by suitable PCR amplification schemes, and to insert them into suitable vectors containing the information necessary for replication or integration, are well known to persons skilled in the art (cf., for instance, Sambrook et al.).

More than one copy of a nucleic acid sequence encoding an enzyme of the present invention may be inserted into the host cell to amplify expression of the nucleic acid sequence. Stable amplification of the nucleic acid sequence can be obtained by integrating at least one additional copy of the sequence into the host cell genome using methods well known in the art and selecting for transformants.

The nucleic acid constructs of the present invention may also comprise one or more nucleic acid sequences which encode one or more factors that are advantageous in the expression of the polypeptide, e.g., an activator (e.g., a trans-acting factor), a chaperone, and a processing protease. Any factor that is functional in the host cell of choice may be used in the present invention. The nucleic acids encoding one or more of these factors are not necessarily in tandem with the nucleic acid sequence encoding the polypeptide.

### Host Cells

The DNA sequence encoding the subtilases and/or subtilase variants of the present invention may be either homologous or heterologous to the host cell into which it is introduced. If homologous to the host cell, i.e. produced by the host cell in nature, it will typically be operably connected to another promoter sequence or, if applicable, another secretory signal sequence and/or terminator sequence than in its natural environment. The term “homologous” is intended to include a DNA sequence encoding an enzyme native to the host organism in question. The term “heterologous” is intended to include a DNA sequence not expressed by the host cell in nature. Thus, the DNA sequence may be from another organism, or it may be a synthetic sequence.

The host cell into which the DNA construct or the recombinant vector of the invention is introduced may be any cell that is capable of producing the present subtilases and/or subtilase variants, such as prokaryotes, e.g. bacteria or eukaryotes, such as fungal cells, e.g. yeasts or filamentous fungi, insect cells, plant cells or mammalian cells.

Examples of bacterial host cells which, on cultivation, are capable of producing the subtilases or subtilase variants of the invention are gram-positive bacteria such as strains of Bacillus, e.g. strains of B. subtilis, B. licheniformis, B. lentus, B. brevis, B. stearothermophilus, B. alkalophilus, B. amyloliquefaciens, B. coagulans, B. circulans, B. lautus, B. megaterium or B. thuringiensis, or strains of Streptomyces, such as S. lividans or S. murinus, or gram-negative bacteria such as Escherichia coli or Pseudomonas sp.

The transformation of the bacteria may be effected by protoplast transformation, electroporation, conjugation, or by using competent cells in a manner known per se (cf. Sambrook et al., supra).

When expressing the subtilases and/or subtilase variant in bacteria such as E. coli, the enzyme may be retained in the cytoplasm, typically as insoluble granules (known as inclusion bodies), or it may be directed to the periplasmic space by a bacterial secretion sequence. In the former case, the cells are lysed and the granules are recovered and denatured after which the enzyme is refolded by diluting the denaturing agent. In the latter case, the enzyme may be recovered from the periplasmic space by disrupting the cells, e.g. by sonication or osmotic shock, to release the contents of the periplasmic space and recovering the enzyme.

When expressing the subtilases and/or subtilase variant in gram-positive bacteria such as Bacillus or Streptomyces strains, the enzyme may be retained in the cytoplasm, or it may be directed to the extracellular medium by a bacterial secretion sequence. In the latter case, the enzyme may be recovered from the medium as described below.

Examples of host yeast cells include cells of a species of Candida, Kluyveromyces, Saccharomyces, Schizosaccharomyces, Candida, Pichia, Hansenula, or Yarrowia. In a particular embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. Other useful yeast host cells are a Kluyveromyces lactis Kluyveromyces fragilis Hansenula polymorpha, Pichia pastoris Yarrowia lipolytica, Schizosaccharomyces pombe, Ustilgo maylis, Candida maltose, Pichia guillermondii and Pichia methanolio cell (cf. Gleeson et al., J. Gen. Microbiol. 132, 1986, pp. 3459-3465; U.S. Pat. No. 4,882,279 and U.S. Pat. No. 4,879,231). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathern et al., editors, 1981). Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153:163; and Hinnen et al., 1978, Proceedings of the National Academy of Sciences USA 75:1920.

Examples of filamentous fungal cells include filamentous forms of the subdivision Eumycota and Oomycota (as defined by Hawksworth et al., 1995, supra), in particular it may of the a cell of a species of Acremonium, such as A. chrysogenum, Aspergillus, such as A. awamori, A. foetidus, A. japonicus, A. niger, A. nidulans or A. oryzae, Fusarium, such as F. bactridioides, F. cerealis, F. crookwellense, F. culmorum, F. graminearum, F. graminum, F. heterosporum, F. negundi, F. reticulatum, F. roseum, F. sambucinum, F. sarcochroum, F. sulphureum, F. trichothecioides or F. oxysporum, Humicola, such as H. insolens or H. lanuginose, Mucor, such as M. miehei, Myceliophthora, such as M. thermophilum, Neurospora, such as N. crassa, Penicillium, such as P. purpurogenum, Thielavia, such as T. terrestris, Tolypocladium, or Trichoderma, such as T. harzianum, T. koningii, T. longibrachiatum, T. reesei or T. viride, or a teleomorph or synonym thereof. The use of Aspergillus spp. for the expression of proteins is described in, e.g., EP 272 277, EP 230 023.

Examples of insect cells include a Lepidoptera cell line, such as Spodoptera frugiperda cells or Trichoplusia ni cells (cf. U.S. Pat. No. 5,077,214). Culture conditions may suitably be as described in WO 89/01029 or WO 89/01028. Transformation of insect cells and production of heterologous polypeptides therein may be performed as described in U.S. Pat. No. 4,745,051; U.S. Pat. No. 4,775,624; U.S. Pat. No. 4,879,236; U.S. Pat. No. 5,155,037; U.S. Pat. No. 5,162,222; EP 397,485).

Examples of mammalian cells include Chinese hamster ovary (CHO) cells, HeLa cells, baby hamster kidney (BHK) cells, COS cells, or any number of other immortalized cell lines available, e.g., from the American Type Culture Collection. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Ausubel et al., Current Protocols in Molecular Biology, John Wiley and Sons, Inc., N.Y., 1987, Hawley-Nelson et al., Focus 15 (1993), 73; Ciccarone et al., Focus 15 (1993), 80; Graham and van der Eb, Virology 52 (1973), 456; and Neumann et al., EMBO J. 1 (1982), 841-845. Mammalian cells may be transfected by direct uptake using the calcium phosphate precipitation method of Graham and Van der Eb (1978, Virology 52:546).

### Methods for Expression and Isolation of Proteins

To express an enzyme of the present invention the above mentioned host cells transformed or transfected with a vector comprising a nucleic acid sequence encoding an enzyme of the present invention are typically cultured in a suitable nutrient medium under conditions permitting the production of the desired molecules, after which these are recovered from the cells, or the culture broth.

The medium used to culture the host cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The media may be prepared using procedures known in the art (see, e.g., references for bacteria and yeast; Bennett, J. W. and LaSure, L., editors, More Gene Manipulations in Fungi, Academic Press, CA, 1991).

If the enzymes of the present invention are secreted into the nutrient medium, they may be recovered directly from the medium. If they are not secreted, they may be recovered from cell lysates. The enzymes of the present invention may be recovered from the culture medium by conventional procedures including separating the host cells from the medium by centrifugation or filtration, precipitating the proteinaceous components of the supernatant or filtrate by means of a salt, e.g. ammonium sulfate, purification by a variety of chromatographic procedures, e.g. ion exchange chromatography, gelfiltration chromatography, affinity chromatography, or the like, dependent on the enzyme in question.

The enzymes of the invention may be detected using methods known in the art that are specific for these proteins. These detection methods include use of specific antibodies, formation of a product, or disappearance of a substrate. For example, an enzyme assay may be used to determine the activity of the molecule. Procedures for determining various kinds of activity are known in the art.

The enzymes of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J-C Janson and Lars Ryden, editors, VCH Publishers, New York, 1989).

When an expression vector comprising a DNA sequence encoding an enzyme of the present invention is transformed/transfected into a heterologous host cell it is possible to enable heterologous recombinant production of the enzyme. An advantage of using a heterologous host cell is that it is possible to make a highly purified enzyme composition, characterized in being free from homologous impurities, which are often present when a protein or peptide is expressed in a homologous host cell. In this context homologous impurities mean any impurity (e.g. other polypeptides than the enzyme of the invention) which originates from the homologous cell where the enzyme of the invention is originally obtained from.

### Commercial Enzyme Applications

The present invention also relates to compositions comprising subtilase and/or subtilase variants of the present invention. For example the subtilase/subtilase variant may be used in compositions for personal care, such as shampoo, soap bars, skin lotion, skin cream, hair dye, toothpaste, contact lenses, cosmetics, toiletries, or in compositions used for treating textiles, for manufacturing food, e.g. baking or feed, or in compositions for cleaning purposes, e.g. detergents, dishwashing compositions or for cleaning hard surfaces.

### Detergents

The subtilase and/or subtilase variant of the invention may for example be used in detergent composition. It may be included in the detergent composition in the form of a non-dusting granulate, a stabilized liquid, or a protected enzyme. Non-dusting granulates may be produced, e.g., as disclosed in U.S. Pat. Nos. 4,106,991 and 4,661,452 and may optionally be coated by methods known in the art. Examples of waxy coating materials are poly(ethylene oxide) products (polyethylene glycol, PEG) with mean molecular weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in patent GB 1483591. Liquid subtilase/subtilase variant preparations may, for instance, be stabilized by adding a polyol such as propylene glycol, a sugar or sugar alcohol, lactic acid or boric acid according to established methods. Other enzyme stabilizers are well known in the art. Protected subtilase/subtilase variants may be prepared according to the method disclosed in EP 238,216.

The detergent composition may be in any convenient form, e.g. as powder, granules, paste or liquid. A liquid detergent may be aqueous, typically containing up to 70% water and 0-30% organic solvent, or non-aqueous.

The detergent composition may comprise one or more surfactants, each of which may be anionic, nonionic, cationic, or zwitterionic. The detergent will usually contain 0-50% of anionic surfactant such as linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. It may also contain 0-40% of nonionic surfactant such as alcohol ethoxylate (AEO or AE), carboxylated alcohol ethoxylates, nonylphenol ethoxylate, alkylpolyglycoside, alkyldimethylamine oxide, ethoxylated fatty acid monoethanolamide, fatty acid monoethanolamide, or polyhydroxy alkyl fatty acid amide (e.g. as described in WO 92/06154).

The detergent composition may additionally comprise one or more other enzymes, such as e.g. proteases, amylases, lipolytic enzymes, cutinases, cellulases, peroxidases, oxidases, and further anti-microbial polypeptides.

The detergent may contain 1-65% of a detergent builder or complexing agent such as zeolite, diphosphate, triphosphate, phosphonate, citrate, nitrilotriacetic acid (NTA), ethylenediaminetetraacetic acid (EDTA), diethylenetriaminepentaacetic acid (DTMPA), alkyl- or alkenylsuccinic acid, soluble silicates or layered silicates (e.g. SKS-6 from Hoechst). The detergent may also be unbuilt, i.e. essentially free of detergent builder.

The detergent may comprise one or more polymers. Examples are carboxymethylcellulose (CMC), poly(vinylpyrrolidone) (PVP), polyethyleneglycol (PEG), poly(vinyl alcohol) (PVA), polycarboxylates such as polyacrylates, maleic/acrylic acid copolymers and lauryl methacrylate/acrylic acid copolymers.

The detergent may contain a bleaching system which may comprise a H₂O₂ source such as perborate or percarbonate which may be combined with a peracid-forming bleach activator such as tetraacetylethylenediamine (TAED) or nonanoyloxybenzenesulfon-ate (NOBS). Alternatively, the bleaching system may comprise peroxyacids of, e.g., the amide, imide, or sulfone type.

The detergent composition may be stabilized using conventional stabilizing agents, e.g. a polyol such as propylene glycol or glycerol, a sugar or sugar alcohol, lactic acid, boric acid, or a boric acid derivative such as, e.g., an aromatic borate ester, and the composition may be formulated as described in, e.g., WO 92/19709 and WO 92/19708.

The detergent may also contain other conventional detergent ingredients such as, e.g., fabric conditioners including clays, foam boosters, suds suppressors, anti-corrosion agents, soil-suspending agents, anti-soil-redeposition agents, dyes, bactericides, optical brighteners, or perfume.

The pH (measured in aqueous solution at use concentration) will usually be neutral or alkaline, e.g. in the range of 7-11.

### Dishwashing Composition

Furthermore, the subtilases and/or subtilase variants of the present invention may also be used in dishwashing detergents.

Dishwashing detergent compositions typically comprise a surfactant which may be anionic, non-ionic, cationic, amphoteric or a mixture of these types. The detergent may contain 0-90% of non-ionic surfactant such as low- to non-foaming ethoxylated propoxylated straight-chain alcohols.

The detergent composition may contain detergent builder salts of inorganic and/or organic types. The detergent builders may be subdivided into phosphorus-containing and non-phosphorus-containing types. The detergent composition usually contains 1-90% of detergent builders.

Examples of phosphorus-containing inorganic alkaline detergent builders, when present, include the water-soluble salts especially alkali metal pyrophosphates, orthophosphates, and polyphosphates. An example of phosphorus-containing organic alkaline detergent builder, when present, includes the water-soluble salts of phosphonates. Examples of non-phosphorus-containing inorganic builders, when present, include water-soluble alkali metal carbonates, borates and silicates as well as the various types of water-insoluble crystalline or amorphous alumino silicates of which zeolites are the best-known representatives.

Examples of suitable organic builders include the alkali metal, ammonium and substituted ammonium, citrates, succinates, malonates, fatty acid sulphonates, carboxymetoxy succinates, ammonium polyacetates, carboxylates, polycarboxylates, am inopolycarboxylates, polyacetyl carboxylates and polyhydroxsulphonates.

Other suitable organic builders include the higher molecular weight polymers and copolymers known to have builder properties, for example appropriate polyacrylic acid, polymaleic and polyacrylic/polymaleic acid copolymers and their salts.

The dishwashing detergent composition may contain bleaching agents of the chlorine/bromine-type or the oxygen-type. Examples of inorganic chlorine/bromine-type bleaches are lithium, sodium or calcium hypochlorite and hypobromite as well as chlorinated trisodium phosphate. Examples of organic chlorine/bromine-type bleaches are heterocyclic N-bromo and N-chloro imides such as trichloroisocyanuric, tribromoisocyanuric, dibromoisocyanuric and dichloroisocyanuric acids, and salts thereof with water-solubilizing cations such as potassium and sodium. Hydantoin compounds are also suitable.

The oxygen bleaches may be in the form of an inorganic persalt, particularly with a bleach precursor or as a peroxy acid compound. Examples of suitable peroxy bleach compounds include alkali metal perborates, e.g. tetrahydrates and monohydrates, alkali metal percarbonates, persilicates and perphosphates. Particularly activator materials may be TAED and glycerol triacetate.

The dishwashing detergent composition may be stabilized using conventional stabilizing agents for enzymes, e.g. a polyol such as e.g. propylene glycol, a sugar or a sugar alcohol, lactic acid, boric acid, or a boric acid derivative, e.g. an aromatic borate ester.

The dishwashing detergent composition may also contain other conventional detergent ingredients, e.g. deflocculant material, filler material, foam depressors, anti-corrosion agents, soil-suspending agents, sequestering agents, anti-soil redeposition agents, dehydrating agents, dyes, bactericides, fluorescers, thickeners and perfumes.

Finally, the subtilases and/or subtilase variants of the invention may be used in conventional dishwashing-detergents, e.g. in any of the detergents described in any of the following patent publications:

EP 518719, EP 518720, EP 518721, EP 516553, EP 516554, EP 516555, GB 2200132, DE 3741617, DE 3727911, DE 4212166, DE 4137470, DE 3833047, WO 93/17089, DE 4205071, WO 52/09680, WO 93/18129, WO 93/04153, WO 92/06157, WO 92/08777, EP 429124, WO 93/21299, U.S. Pat. No. 5,141,664, EP 561452, EP 561446, GB 2234980, WO 93/03129, EP 481547, EP 530870, EP 533239, EP 554943, EP 346137, U.S. Pat. No. 5,112,518, EP 318204, EP 318279, EP 271155, EP 271156, EP 346136, GB 2228945, CA 2006687, WO 93/25651, EP 530635, EP 414197, U.S. Pat. No. 5,240,632.

### Personal Care Applications

Another useful application area for the subtilases and/or subtilase variants of the present invention is the personal care area where the end-user is in close contact with the protein, and where certain problems with allergenicity has been encountered in experimental setups (Kelling et al., J. All. Clin. Imm., 1998, Vol. 101, pp. 179-187 and Johnston et al., Hum. Exp. Toxicol., 1999, Vol.18, p. 527).

First of all the conjugate or compositions of the invention can advantageously be used for personal care products, such as hair care and hair treatment products. This include products such as shampoo, balsam, hair conditioners, hair waving compositions, hair dyeing compositions, hair tonic, hair liquid, hair cream, shampoo, hair rinse, hair spray.

Further contemplated are oral care products such as dentifrice, oral washes, chewing gum.

Also contemplated are skin care products and cosmetics, such as skin cream, skin milk, cleansing cream, cleansing lotion, cleansing milk, cold cream, cream soap, nourishing essence, skin lotion, milky lotion, calamine lotion, hand cream, powder soap, transparent soap, sun oil, sun screen, shaving foam, shaving cream, baby oil lipstick, lip cream, creamy foundation, face powder, powder eye-shadow, powder, foundation, make-up base, essence powder, whitening powder.

Also for contact lenses hygiene products the subitlases and/or subtilase variants of the invention may be used advantageously. Such products include cleaning and disinfection products for contact lenses.

### Food and Feed

The subtilase variants and/or subtilases of the present invention may also be used in food or feed products. For example said subtilase variants/subtilases may be used modify the gluten phase of the dough, e.g. a hard wheat flour can be softened with a protease. Another example is within the brewery industry, where said subtilase variants/subtilases may be used for brewing with unmalted cereals and/or for controlling the nitrogen content.

Within the animal feed industry said subtilase variants and/or subtilases may be used for so to speak expanding the animals' digestion system.

### Materials and Methods

### Materials

### ELISA Reagents:

Horse Radish Peroxidase labelled pig anti-rabbit-Ig (Dako, DK, P217, dilution 1:1000).
Mouse anti-rat IgE (Serotec MCA193; dilution 1:200).
Biotin-labelled mouse anti-rat IgG1 monoclonal antibody (Zymed 03-9140; dilution 1:1000)
Biotin-labelled rat anti-mouse IgG1 monoclonal antibody (Serotec MCA336B; dilution 1:2000)
Streptavidin-horse radish peroxidase (Kirkegård & Perry 14-30-00; dilution 1:1000).
OPD: o-phenylene-diamine, (Kementec cat no. 4260)
Rabbit anti-Savinase polyclonal IgG prepared by conventional means
Rat anti-Savinase polyclonal IgE prepared by conventional means.

### Buffers and Solutions:

PBS (pH 7.2 (1 liter))

| | |
|---|---|
| NaCl | 8.00 g |
| KCl | 0.20 g |
| K₂HPO₄ | 1.04 g |
| KH₂PO₄ | 0.32 g |

Succinyl-Alanine-Alanine-Proline-Phenylalanine-paranitro-anilide (Suc-AAPF-pNP) Sigma no. S-7388, Mw 624.6 g/mol.

### Methods

Measurement of the Concentration of Specific IgE in the s.c. Mouse Model by ELISA

The relative concentrations of specific IgE serum antibodies in the mice produced in response to s.c. injection of proteins are measured by a three layer sandwich ELISA according to the following procedure:
1) The ELISA-plate was coated with 10 microgram rat anti-mouse IgE (Serotech MCA419; dilution 1:100) Buffer 1 (50 microL/well). Incubated over night at 4° C.
2) The plates were emptied and blocked with 2 (wt/v)% skim milk, PBS for at least ½ hour at room temperature (200 microL/well). Gently shaken. The plates were washed 3 times with 0.05 (v/v)% Tween20, PBS.
3) The plates were incubated with mouse sera (50 microL/well), starting from undiluted and continued with 2-fold dilutions. Some wells were kept free for buffer 4 only (blanks). Incubated for 30 minutes at room temperature. Gently shaken. The plates were washed 3 times in 0.05 (v/v)% Tween20, PBS.
4) The subtilase or subtilase variant was diluted in 0.05 (v/v)% Tween20, 0.5 (wt/v)% skim milk, PBS to the appropriate protein concentration. 50 microl/well was incubated for 30 minutes at room temperature. Gently shaken. The plates were washed 3 times in 0.05 (v/v)% Tween20, PBS.
5) The specific polyclonal anti-subtilase or anti-subtilase variant antiserum serum (plg) for detecting bound antibody was diluted in 0.05 (v/v)% Tween20, 0.5 (wt/v)% skim milk, PBS. 50 microl/well was incubated for 30 minutes at room temperature. Gently shaken. The plates were washed 3 times in 0.05 (v/v)% Tween20, PBS.
6) Horseradish Peroxidase-conjugated anti-plg-antibody was diluted in 0.05 (v/v)% Tween20, 0.5 (wt/v)% skim milk, PBS. 50 microl/well was incubated at room temperature for 30 minutes. Gently shaken. The plates were washed 3 times in 0.05 (v/v)% Tween20, PBS.
7) 0.6 mg ODP/ml+0.4 microL H₂O₂/ml were mixed in Citrate buffer pH 5.2.
8) The solution was made just before use and incubated for 10 minutes.
9) 50 microl/well.
10) The reaction was stopped by adding 50 microl 2 N H₂SO₄/well.
11) The plates were read at 492 nm with 620 nm as reference.

Similar determination of IgG can be performed using anti mouse-IgG and standard rat IgG reagents.

### Measurement of the Concentration of Specific IqE in the MINT Assay by ELISA

The relative concentrations of specific IgE serum antibodies in the mice produced in response to intranasal dosing of proteins are measured by a three layer sandwich ELISA according to the following procedure:
1) The ELISA-plate (Nunc Maxisorp) was coated with 100 microliter/well rat anti-mouse IgE Heavy chain (HD-212-85-IgE3 diluted 1:100 in 0.05 M Carbonate buffer pH 9.6). Incubated over night at 4° C.
2) The plates were emptied and blocked with 200 microliter/well 2% skim milk in 0.15 M PBS buffer pH 7.5 for 1 hour at 4° C. The plates were washed 3 times with 0.15 M PBS buffer with 0.05% Tween20.
3) The plates were incubated with dilutions of mouse sera (100 microL/well), starting from an 8-fold dilution and 2-fold dilutions hereof in 0.15 M PBS buffer with 0.5% skim milk and 0.05% Tween20. Appropriate dilutions of positive and negative control serum samples plus buffer controls were included. Incubated for 1 hour at room temperature. Gently shaken. The plates were washed 3 times in 0.15 M PBS buffer with 0.05% Tween20.
4) 100 microliter/well of subtilase or subtilase variant diluted to 1 microgram protein/ml in 0.15 M PBS buffer with 0.5% skim milk and 0.05% Tween20 was added to the plates. The plates were incubated for 1 hour at 4° C. The plates were washed 3 times with 0.15 M PBS buffer with 0.05% Tween20.
5) The specific polyclonal anti-subtilase or anti-subtilase variant antiserum serum (plg) for detecting bound antigen was diluted in 0.15 M PBS buffer with 0.15% skim milk and 0.05% Tween20. 100 microl/well was incubated for 1 hour at 4° C. The plates were washed 3 times in 0.15 M PBS buffer with 0.05% Tween20.
6) 100 microliter/well pig anti-rabbit Ig conjugated with peroxidase diluted 1:1000 in 0.15 M PBS buffer with 0.5% skim milk and 0.05% Tween20 was added to the plates. Incubated for 1 hour at 4° C. The plates were washed 3 times in 0.15 M PBS buffer with 0.05% Tween20.
7) 250 microliter/well 0.1 M Citrat/phosphat buffer pH 5.0 was added to the plates. Incubated for approximately 1 minute. The plates were emptied.
8) 100 microliter/well ortho-phenylenediamine (OPD) solution (10 mg OPD diluted in 12.5 ml Citrat/phosphat buffer pH 5.0 and 12.5 microliter 30% hydrogen peroxide added just before use) was added to the plates. Incubation for 4 minutes at room temperature.
9) The reaction was stopped by adding 150 microliter/well 1M H₂SO₄.
10) The plates were read at 490 nm with 620 nm as reference.

### Protein Engineering

The Savinase/subtilase variants were obtained by site-directed mutagenesis of the corresponding nucleic acid sequences as described in for example Sambrook et al. (1989), Molecular Cloning. A Laboratory Manual, Cold Spring Harbour, N.Y.).

### Measurement of Antibody Binding Capability

Activation of CovaLink Plates:

A fresh stock solution of 10 mg/ml cyanuric chloride in acetone is diluted into PBS, while stirring, to a final concentration of 1 mg/ml and immediately aliquoted into CovaLink NH2 plates (Nunc) (100 microliter per well) and incubated for 5 minutes at room temperature. After three washes with PBS, the plates are dried at 50° C. for 30 minutes, sealed with sealing tape, and stored in plastic bags at room temperature for up to 3 weeks.

Immobilization of Antibody/Competitive Antigen:

Activated CovaLink NH2 plates are coated overnight at 4° C. with 100 microliter of the desired protein (5 micro gram/ml) in PBS followed by 30 min incubation with 2 (wt/v)% skim milk, PBS at room temperature and four washes in 0.05 (v/v)% Tween20, PBS.

Protease Activity:

Analysis with Suc-Ala-Ala-Pro-Phe-pNa:

Proteases cleave the bond between the peptide and p-nitroaniline to give a visible yellow colour absorbing at 405 nm. Briefly, 100 mg suc-AAPF-pNa is dissolved into 1 ml dimethyl sulfoxide (DMSO). 100 microliter of this is diluted into 10 ml with Britton and Robinson buffer, pH 8.3 and used as substrate for the protease. Reaction is detected kinetically in a spectrophotometer.

Measurement of Ability to Bind to Anti-Savinase Antibody:

The ability of subtilases/subtilase variants to bind to anti-Savinase antibody was compared with that of Savinase by coating CovaLink NH2 plates with mouse anti-rat IgE monoclonal antibodies and subsequently saturating the antibodies with anti-Savinase specific rat polyclonal IgE. The plates were incubated with antigen, i.e. Savinase (control), subtilases for which the binding ability should be tested (e.g. a subtilase library expressing subtilase variants). The amount of bound antigen was determined by incubation with anti-wild type Savinase polyclonal rabbit antiserum.

Measurement of the Functionality of the Active Site:

A ‘backbone protease’ inhibitor is immobilized in the wells and incubated with an excess of the protein variant and labelled antibodies. The level of bound antibodies is determined.

25 microliter sample and 25 microliter anti-Savinase antibody (both diluted in 0.05 (v/v)% Tween20, PBS with 0.5% (wt/v) skim milk) are added to the coated well and incubated at room temperature (30 min). The supernatant is removed and the wells are washed three times in 0.05 (v/v)% Tween20, PBS.

50 microliter HRP-labelled species-specific anti-Ig antibody is added and incubated 30 min, then the wells are wash three times in 0.05 (v/v)% Tween20, PBS. Finally, 50 microliter ODP-H2O2-mixture is added and A492 is measured kinetically to determine the level of bound antibodies. Dilutions are adjusted such that the ‘backbone protein’ gives none or very little level of bound antibody.

A separate sample is analysed for functionality and the two values are compared.

Desired protein variants show a level of bound antibody at least 2 times higher or 2 times lower (a Delta antibody binding value of at least 2) and at the same time a level of functionality similar to the ‘backbone protein’.

### Examples

### Example 1

### Identification of Epitope Sequences and Epitope Patterns in Savinase.

Epitope sequences and patterns were determined as previously described in WO 01/83559 example 1.

High diversity libraries (10¹²) of phages expressing random hexa-, nona- or do-decapetides as part of their membrane proteins, were screened for their capacity to bind purified specific rabbit IgG, and purified rat and mouse IgG1 and IgE antibodies. The phage libraries were obtained according to prior art (se WO 9215679 hereby incorporated by reference).

The antibodies were raised in the respective animals by subcutaneous, intradermal, or intratracheal injection of selected target proteins (N=75) including Savinase and other subtilases dissolved in phosphate buffered saline (PBS). The respective antibodies were purified from the serum of immunised animals by affinity chromatography using paramagnetic immunobeads (Dynal AS) loaded with pig anti-rabbit IgG, mouse anti-rat IgG1 or IgE, or rat anti-mouse IgG1 or IgE antibodies.

The respective phage libraries were incubated with the IgG, IgG1 and IgE antibody coated beads. Phages, which express oligopeptides with affinity for rabbit IgG, or rat or mouse IgG1 or IgE antibodies, were collected by exposing these paramagnetic beads to a magnetic field. The collected phages were eluted from the immobilised antibodies by mild acid treatment, or by elution with intact enzyme. The isolated phages were amplified as know to the specialist. Alternatively, immobilised phages were directly incubated with E. coli for infection. In short, F-factor positive E. coli (e.g. XL-1 Blue, JM101, TG1) were infected with M13-derived vector in the presence of a helper-phage (e.g. M13K07), and incubated, typically in 2xYT containing glucose or IPTG, and appropriate antibiotics for selection. Finally, cells were removed by centrifugation. This cycle of events was repeated 2-5 times on the respective cell supernatants. After selection round 2, 3, 4, and 5, a fraction of the infected E. coli was incubated on selective 2xYT agar plates, and the specificity of the emerging phages was assessed immunologically. Thus, phages were transferred to a nitrocellulase (NC) membrane. For each plate, 2 NC-replicas were made. One replica was incubated with the selection antibodies, the other replica was incubated with the selection antibodies and the immunogen used to obtain the antibodies as competitor. Those plaques that were absent in the presence of immunogen, were considered specific, and were amplified according to the procedure described above.

The specific phage-clones were isolated from the cell supernatant by centrifugation in the presence of polyethylenglycol. DNA was isolated, the DNA sequence coding for the oligopeptide was amplified by PCR, and the DNA sequence was determined, all according to standard procedures. The amino acid sequence of the corresponding oligopeptide was deduced from the DNA sequence.

Thus, a number of peptide sequences with specificity for the protein specific antibodies, described above, were obtained. These sequences were collected in a database, and analysed by sequence alignment to identify epitope patterns. For this sequence alignment, conservative substitutions (e.g. aspartate for glutamate, lysine for arginine, serine for threonine) were considered as one. This showed that most sequences were specific for the protein the antibodies were raised against. However, several cross-reacting sequences were obtained from phages that went through 2 selection rounds only. In the first round 22 epitope patterns were identified.

In further rounds of phage display, more antibody binding sequences were obtained leading to more epitope patterns. Further, the literature was searched for peptide sequences that have been found to bind environmental allergen-specific antibodies (J All Clin Immunol 93 (1994) pp. 34-43; Int Arch Appl Immunol 103 (1994) pp. 357-364; Clin Exp Allergy 24 (1994) pp. 250-256; Mol Immunol 29 (1992) pp. 1383-1389; J Immunol 121 (1989) pp. 275-280; J. Immunol 147 (1991) pp. 205-211; Mol Immunol 29 (1992) pp. 739-749; Mol Immunol 30 (1993) pp. 1511-1518; Mol Immunol 28 (1991) pp. 1225-1232; J. Immunol 151 (1993) pp. 7206-7213). These antibody binding peptide sequences were included in the database.

These sequences were collected in a database, and analyzed by sequence alignment to identify epitope patterns. For this sequence alignment, conservative substitutions (e.g. aspartate for glutamate, lysine for arginine, serine fro threonine) were considered as one. This showed that most sequences were specific for the protein the antibodies were raised against. However, epitope patterns were shown to be applicable across proteins, antibody-types and animal species. Yet, 75 epitope patterns were identified.

These epitope patterns were automatically assessed on the 3D-structure of Savinase (as described in WO 01/83559) and the number of potential epitopes each amino acid is part of (in the table 1 referred to as frequency) was calculated (table 1).

TABLE 1

| Amino acid position with the given frequency | Frequency |
|---|---|
| 21, 38, 42, 46, 53, 62, 78, 82, 89, 98, 101, | 1 |
| 102, 116, 117, 128, 135, 140, 143, 156, 160, | |
| 162, 191, 197, 211, 212, 248 | |
| 1, 9, 10, 18, 45, 47, 49, 59, 75, 80, 86, 88, 96, | 2 |
| 112, 127, 131, 133, 137, 145, 155, 157, 173, | |
| 183, 185, 188, 189, 210, 213, 242, 245, 253, | |
| 255 | |
| 141, 218, 247 | 3 |
| 22, 52, 104, 130, 172, 181, 195 | 4 |
| 19, 40, 48, 61, 136, 262, 275 | 5 |
| 14, 57, 167, 186, 196 | 6 |
| 15, 20, 50, 109, 129, 161, 272 | 7 |
| 54, 60, 260 | 8 |
| 194 | 9 |
| 55 | 10 |
| 94, 170 | 12 |

### Example 2

### Localisation on the 3-Dimensional Structure of Savinase the Amino Acid Positions Involved in Potential IgE Epitopes

Amino acid positions which were found to be most likely involved in potential IgE epitopes (in general these were amino acids which were found to be potentially involved in at least 3 IgE epitopes) were manually localised on the 3D-structure of Savinase (Protein Data Bank entry 1SVN; Betzel, C., Klupsch, S., Papendorf, G., Hastrup, S., Branner, S., Wilson, K. S.: Crystal structure of the alkaline proteinase Savinase from Bacillus lentus at 1.4 Å resolution. J Mol Biol 223 pp. 427 (1992)), using appropriate software (e.g. SwissProt Pdb Viewer, WebLite Viewer).

By localising the amino acids on the 3-dimensional structure it was found that the amino acids potentially involved in IgE epitopes cluster in 3 major areas:
area 1: P14, A15, R19, G20, T22, A272, R275
area 2: A48, F50, P52, E54, P55, S57, D60, G61, K94, V104, Q109
area 3: P129, S130, E136, N140, S161, Y167, R170, A172, D181, R186, A194, G195, L196, R247, T260, L262
positions P39 and N218 are standing alone.

### Example 3

### Localisation on the 3-Dimensional Structure of Savinase the Amino Acid Positions Selected for Protein Engineering

The amino acids were selected for epitope protein engineering based upon structural and enzyme activity related considerations, meaning that positions suggested by 3D-analysis or experiences from other protein engineering concepts to give beneficial effects on the activity and/or stability of the enzymes, were prioritised.

The selected amino acids are in
area 1: A15, R19, R275
area 2: S57
area 3: E136, N140, Y167, R170, A172, D181, R186, A194, G195, R247, T260, L262,
position N218.

These positions were engineered separately, or in combination with each other. Combinations were selected based upon the performance of the individual mutations, and/or on topographic aspects (covering as large an area as possible with as few mutations as possible).

On the basis of these considerations it was found that the positions and combination of positions shown in table 2 would be relevant to engineer to obtain subtilase with modified immunogenicity/reduced allergenicity.

TABLE 2

| Single | Double | | |
|---|---|---|---|
| position | positions | Triple positions | Quadruple positions |
| A15X | | | |
| R19X | | | |
| S57X | S57X + R170X | S57X + R170X + | |
| | S57X + R247X | R247X | |
| | | S57X + Y167X + | |
| | | R247X | |
| | | S57X + D181X + | |
| | | R247X | |
| E136X | E136X + N140X | | |
| N140X | N140X + A172X | | |
| Y167X | | Y167X + R170X + | Y167X + R170X + |
| | | N218X | A194X + N218X |
| | | Y167X + R170X + | |
| | | A194X | |
| R170X | R170X + N206X | | |
| | R170X + N218X | | |
| D181X | | | |
| R186X | | | |
| G195X | | | |
| R247X | S57X + R247X | S57X + R170X + | |
| | | R247X | |
| T260X | | | |
| L262X | | | |
| R275X | | | |

### Example 4

Testing of Savinase Variants with Reduced Antibody Binding Capacity

Identification of promising variants was performed by assessing changes in the antibody binding capacity of the enzymatically active variants of example 3, expressed in Bacillus spp.

Changes in the antibody binding capacity (Delta-binding) of at least 2-fold were considered significant (P<0.05). The mutations introduced in these variants were identified by DNA sequence analysis using standard methods, e.g. see Molecular cloning: A laboratory manual (Sambrook et al. (1989), Cold Spring Harbor lab., Cold Spring Harbor, N.Y.; Ausubel, F. M. et al. (eds.))

The subtilase variants with a Delta-binding value of at least 2.0 and their antibody binding capacity is shown in table 3

TABLE 3

| | Antibody binding capacity shown as |
|---|---|
| Subtilase variants | Delta-binding |
| N18D | 2.0 |
| S57P + R170L | 2.1 |
| S57P + R170L + R247Q | 2.0 |
| E136R | 2.8 |
| E136R + N140D | 2.1 |
| N140D + A172D | 3.8 |
| Y167I + R170L + N218S | 4.8 |
| Y167I + R170L + A194P + N218S | 3.2 |
| R170F | 2.4 |
| R170L + Q206E | 2.1 |
| D181N | 2.9 |
| R247E | 2.0 |
| R247H | 2.0 |
| R247K | 2.0 |
| R247Q | 2.0 |
| R275E | 2.0 |
| S57P + Y167F + R247Q | 3.0 |
| S57P + R170L + R247Q | 2.1 |

### Example 5

Testing Savinase Variants for Reduced Allergenicity in s.c. Mouse Model

Mice were immunised subcutanuous weekly, for a period of 20 weeks, with 50 microl 0.9% (wt/vol) NaCl (control group), or 50 microl 0.9% (wt/vol) NaCl containing 10 microg of protein. Each group contained 10 female Balb/C mice (about 20 grams) purchased from Bomholdtgaard, Ry, Denmark. Blood samples (100 microl) were collected from the eye every other week before the next immunization. Serum was obtained by blood clothing, and centrifugation.

For each variant and Savinase the sum of IgE levels detected in each mouse of the same group over a 20 week period (the integrated IgE levels) were calculated. For Savinase the integrated IgE level was equalled 100% and for the variants it was calculated according to Savinase. Table 4 shows those variants were the integrated IgE level was at least 33% less than for Savinase, as this was found to be statistically different from Savinase.

TABLE 4

| Variants | % integrated IgE compared to Savinase |
|---|---|
| S57P + R170L | 13 |
| S57P + R170L + R247Q | 5 |
| Y167I + R170L + N218S | 15 |
| R170F | 11 |
| D181N | 17 |
| R247E | 30 |
| R247H | 26 |
| R247Q | 17 |

### Example 6

### Testing of Savinase Variants for Reduced Allergenicity In Vivo (MINT Assay).

Mouse intranasal (MINT) model (Robinson et al., Fund. Appl. Toxicol. 34, pp. 15-24, 1996). Mice were dosed intranasally with the proteins on the first and third day of the experiment and from thereon on a weekly basis for a period of 6 weeks. Blood samples were taken 15, 31 and 45 days after the start of the study. Serum was subsequently analysed for IgG1 or IgE levels.

The variants S57P+R170L+R247Q; S57P+R247Q and S221C (inactive) were compared to Alcalase® and Savinase®(in 0.9% NaCl).

The mean titres indicated in the Table 5:

The IgG1 and IgE titres are expressed as the reciprocal of the highest dilution giving a positive ELISA reading converted to log2. A reading is regarded as positive if higher than the OD-mean+2× standard deviation of the negative controls. There were 6 mice per dose level and the results are expressed as group mean titres.

TABLE 5
IgG1 Day 15

| Dose, (μg | | S57P + | | | |
|---|---|---|---|---|---|
| protein/ | | R170L + | | | |
| animal) | Alcalase | R247Q | S221C | S57P + R247Q | Savinase |
| 10 | n.d. | 1.76 | 2 | n.d. | n.d. |
| 3 | 14.83 | 0 | 0 | 3.83 | 3 |
| 1 | 5.83 | 0 | 0.5 | 0.83 | 0 |
| 0.3 | 1.17 | 0 | 0 | 0 | 0 |
| 0.1 | 0 | 0 | 1 | 0.5 | 0 |
| 0.03 | 0 | n.d. | n.d. | 1.17 | 0 |

| | | | | | |
|---|---|---|---|---|---|
| | | S57P + | | | |
| | | R170L + | | | |
| Dose | Alcalase | R247Q | S221C | S57P + R247Q | Savinase |

IgE Day 31

| | | | | | |
|---|---|---|---|---|---|
| 10 | n.d. | 4.17 | 5.67 | n.d. | n.d. |
| 3 | 8 | 3.33 | 2.33 | 5.5 | 7.33 |
| 1 | 5.67 | 5.17 | 3.67 | 6 | 4 |
| 0.3 | 4 | 0 | 3 | 2.33 | 0 |
| 0.1 | 0 | 0 | 0.5 | 0 | 0 |
| 0.03 | 0 | n.d. | n.d. | 0 | 0 |

IgE Day 45

| | | | | | |
|---|---|---|---|---|---|
| 10 | n.d. | 5.5 | 3.67 | n.d. | n.d. |
| 3 | 9.5 | 5.5 | 3.5 | 7.5 | 8.5 |
| 1 | 9.5 | 6.17 | 4.33 | 5.33 | 8.17 |
| 0.3 | 5.83 | 3.67 | 5.17 | 2 | 0.5 |
| 0.1 | 1.50 | 0 | 1.17 | 0.83 | 0 |
| 0.03 | 0 | n.d. | n.d. | 0 | 0 |

n.d. = not determined

From Table 5 it can be concluded that the variants S57P+R170L+R247Q, S221C and S57P+R247Q have considerably less potential for eliciting the production of antigen specific IgG1 and IgE antibody than those of the benchmark proteins, Alcalase and Savinase.

### Example 7

### Test of the Wash Performance of Savinase Variants

The following example provides results from a number of washing tests that were conducted under the conditions indicated.

The detergents are commercial detergents which are inactivated by making a detergent solution and heat it for 5 min. at 85 C in the microwave oven.
pH is “as is” in the current detergent solution and is not adjusted.
Water hardness was adjusted by adding CaCl2*2H2O; MgCl₂*6H2O; NaHCO₃ (Ca²⁺:Mg²⁺:HCO3-=2:1:6) to milli-Q water.

The wash conditions were:
1) Inactivated commercial Tide powder 1 g/l, 30C, 12 min wash, 6 dH.
2) Inactivated commercial Tide liquid 1.5 g/l, 30C, 12 min wash, 6 dH.

The test material is polyester/cotton swatches soiled with blood/milk/carbon black.

After wash the reflectance (R) of the test test material was measured at 460 nm using a J&M Tidas MMS spektrophotometer. The measurements were done according to the manufacturers' protocol.
R_{Variant}: Reflectance of test material washed with variant
R_{Blank}: Reflectance of test material washed with no enzyme
ΔReflectance Rvariant-Rblank

The higher the ΔReflectance the better is the wash performance. The ΔReflectance is calculated for the dosage 5 nM enzyme.

Table 6 shows the results of the wash performance in Tide powder detergent of the 4 Subtilase variants revealing the lowest allergenicity (in terms of IgE production) in mice.

TABLE 6

| Variant | Δ Reflectance | Performance |
|---|---|---|
| Blank | 0.0 | |
| Savinase | 5.0 | |
| R170F | 6.4 | 2 |
| S57P + R170L | 7.0 | 2 |
| S57P + R170L + R247Q | 8.9 | 2 |
| Y167I + R170L + N218S | 7.3 | 2 |

Table 7 shows the results of the wash performance in Tide liquid detergent of the 4 Subtilase variant having the lowest allergenicity (in terms of IgE production) in mice.

TABLE 7

| Variant | Δ Reflectance | Performance |
|---|---|---|
| Blank | 0.0 | |
| Savinase | 3.5 | |
| R170F | 3.5 | 0 |
| S57P + R170L | 4.3 | 2 |
| S57P + R170L + R247Q | 4.8 | 2 |
| Y167I + R170L + N218S | 5.4 | 2 |

```
Performance:
−1: Worse than Savinase
  0: Similar to Savinase
  1: Better than Savinase
  2: Much better than Savinase
```

1-40. (canceled)

41. An isolated subtilase of SEQ ID NO: 1, wherein the Xaa residue
in position 3 is S or T,
in position 4 is V or I,
in position 27 is K or R,
in position 55 is G, A, V, L, I, T, C, M, P, D, N, E, Q, K, R, H, F, Y, W or absent
in position 74 is N or D,
in position 85 is S or N,
in position 97 is S or D,
in position 99 is S, G or R,
in position 101 is S or A,
in position 102 is V, N, Y or I,
in position 121 N or S,
in position 157 is G, D or S,
in position 188 is A or P,
in position 193 is V or M,
in position 199 is V or I,
in position 211 is L or D,
in position 216 is M or S,
in position 226 is A or V,
in position 230 is Q or H,
in position 239 is Q or R,
in position 242 is N or D,
in position 246 is N or K,
in position 268 is T or A,
and wherein the Xaa residues in positions 164, 175 and 241 are one of the following combinations
a) the Xaa in position 164 is G, A, V, L, I, S, T, C, M, P, D, N, E, Q, K, H, F, Y, W or absent, the Xaa in position 175 is G, A, V, L, I, S, T, C, M, P, D, N, E, Q, K, R, H, F, Y, W or absent and the Xaa in position 241 is G, A, V, L, I, S, T, C, M, P, D, N, E, Q, K, R, H, F, Y, W, or absent
or
b) the Xaa in position 164 is G, A, V, L, I, S, T, C, M, P, D, N, E, Q, K, R, H, F, Y, W or absent, the Xaa in position 175 is G, A, V, L, I, S, T, C, M, P, N, E, Q, K, R, H, F, Y, W or absent and the Xaa in position 241 is G, A, V, L, I, S, T, C, M, P, D, N, E, Q, K, R, H, F, Y, W or absent
or
c) the Xaa in position 164 is G, A, V, L, I, S, T, C, M, P, D, N, E, Q, K, R, H, F, Y, W or absent, the Xaa in position 175 is G, A, V, L, I, S, T, C, M, P, D, N, E, Q, K, R, H, F, Y, W or absent and the Xaa in position 241 is G, A, V, L, I, S, T, C, M, P, D, N, E, Q, K, H, F, Y, W or absent.

42. The subtilase of claim 41, wherein the Xaa residue in position 55 is one of the residues: P, K, L, A, W, R, H, C, D, I.

43. The subtilase of claim 41, wherein the Xaa residue in position 164 is one of the residues: G, A, V, L, I, S, T, C, M, P, D, N, E, Q, K, H, F, Y, W or absent.

44. The subtilase of claim 41, wherein the Xaa residue in position 175 is one of the residues: G, A, V, L, I, S, T, C, M, P, N, E, Q, K, R, H, F, Y, W or absent.

45. The subtilase of claim 41, wherein the Xaa residue in position 241 is one of the residues: A, C, D, E, G, H, I, K, L, M, N, P, Q, S, T, V, F, Y.

46. The subtilase of claim 41, wherein the Xaa residue in position 55 is one of the residues: P, K, L, A, W, R, H, C, D, I and the Xaa in position 164 is one of the residues: C, F, G, I, M, N, P, Q, S, T, V, W, Y, A, L, E, D, K, H and the Xaa in position 241 is one of the residues: A, C, D, E, G, H, I, K, L, M, N, P, Q, S, T, V, F, Y.

47. The subtilase of claim 41, wherein the Xaa residue in position 55 is one of the residues: P, K, L, A, W, R, H, C, D, I and the Xaa in position 175 is one of the residues: A, C, F, G, H, I, K, L, M, N, P, Q, R, S, T, V, Y, E, W and the Xaa position 241 is one of the residues: A, C, D, E, G, H, I, K, L, M, N, P, Q, S, T, V, F, Y.

48. The subtilase of claim 41, wherein the Xaa residue in position 55 is P and the Xaa in position 164 is L.

49. The subtilase of claim 41, wherein the Xaa residue in position 55 is P and the Xaa in position 164 is L and the Xaa in position 241 is Q.

50. An isolated subtilase variant comprising a modification at position 57 in combination with a modification in at least one of the positions 181 and 247, using the numbering corresponding to the positions of SEQ ID NO: 2, and wherein the subtilase variant has protease activity.

51. The variant of claim 50, wherein the modification in position 57 is a deletion or a substitution to one of the residues: P, K, L, A, W, R, H, C, D, I.

52. The variant of claim 50, wherein the modification in position 181 is a deletion or a substitution to one of the residues: A, C, F, G, H, I, K, L, M, N, P, Q, R, S, T, V, Y, E, W.

53. The variant of claim 50, wherein the modification in position 247 is a deletion or a substitution to one of the residues: A, C, D, E, G, H, I, K, L, M, N, P, Q, S, T, V, F, Y.

54. The variant of claim 50, the variant being X57P, K, L, A, W, R, H, C, D, I+X181A, C, F, G, H, I, K, L, M, N, P, Q, R, S, T, V, Y, E, W.

55. The variant of claim 50, the variant being X57P, K, L, A, W, R, H, C, D, I+X247A, C, D, E, G, H, I, K, L, M, N, P, Q, S, T, V, F, Y.

56. The variant of claim 50, the variant being X57P, K, L, A, W, R, H, C, D, I+X170C, F, G, I, M, N, P, Q, S, T, V, W, Y, A, L, E, D, K, H+X247A, C, D, E, G, H, I, K, L, M, N, P, Q, S, T, V, F, Y.

57. The variant of claim 50, the variant being X57P, K, L, A, W, R, H, C, D, I+X181A, C, F, G, H, I, K, L, M, N, P, Q, R, S, T, V, Y, E, W+X247A, C, D, E, G, H, I, K, L, M, N, P, Q, S, T, V, F, Y.

58. The variant of claim 50, wherein the variant is one of the following: X57P+X181N, X57P+X247E, X57P+X247H, X57P+X247K, X57P+X247Q, X57P+X170F+X247E, X57P+X170F+X247H, X57P+X170F+X247K, X57P+X170F+X247Q, X57P+X170L+X247E, X57P+X170L+X247H, X57P+X170L+X247K, X57P+X170L+X247Q, X57P+X181N+X247E, X57P+X181N+X247H, X57P+X181N+X247K, X57P+X181N+X247Q.


SEQUENCE LISTING (2 sequences)

SEQ ID NO: 1 - protein, 269 aa, Bacillus lentus
  MISC_FEATURE (3)..(3): Xaa is Ser or Thr
AQXXPWGISRVQAPAAHNRGLTGSGVXVAVLDTGISTHPDLNIRGGASFVPGEPXTQDGNGHGTHVAGTIAALXNSIGVLGVAPXAELYAVKVLGAXGXGXXSSIAQGLEWAGNNGMHVAXLSLGSPSPSATLEQAVNSATSRGVLVVAASGNSGAXSISYPAXYANAMAVGATXQNNNRASFSQYGXGLDIXAPGVNXQSTYPGSTYASXNGTSMATPHVAGAAXLVKXKNPSWSNVXIXXHLKXTATSLGSTNLYGSGLVNAEAAXR

SEQ ID NO: 2 - protein, 275 aa, Bacillus [sequence omitted]